(19) United States
(12) Patent Application Publication
(10) Pub. No.: US 2024/0087704 A1
(43) Pub. Date: Mar. 14, 2024

(54) METHOD AND APPARATUS FOR AUTOMATING MODELS FOR INDIVIDUALIZED ADMINISTRATION OF MEDICAMENTS

(71) Applicants: Christopher Vincent RACKAUCKAS, Cambridge, MA (US); Vijay IVATURI, Centreville, VA (US)

(72) Inventors: Christopher Vincent RACKAUCKAS, Cambridge, MA (US); Vijay IVATURI, Centreville, VA (US)

(21) Appl. No.: 18/271,884

(22) PCT Filed: Jan. 13, 2022
(86) PCT No.: PCT/US2022/012256

§ 371 Date: Jul. 12, 2023

Related U.S. Application Data
(60) Provisional application No. 63/136,719, filed on Jan. 13, 2021.

(51) Int. Cl. G16H 20/10; G16H 50/20; G16H 50/70

(52) U.S. Cl. CPC G16H 20/10; G16H 50/20; G16H 50/70

(57) ABSTRACT

Techniques for generating a dosing protocol for an individual include receiving first data that indicates, for a dose response to a medicament, a non-linear mixed effects (NLME) model of a population with at least one distribution parameter characterizing variations in the population based on an observable property of individuals within the population. At least one of a structural model or a dynamical model of the NLME model is based on training weights of a universal approximator on a least a subset of the population. A candidate dose regimen is evaluated for an expected response by a subject based on the NLME model and one or more properties of the subject. When the expected response is therapeutic, the candidate dose regime of the medicament is administered to the subject.

### CROSS-REFERENCE TO RELATED APPLICATIONS

This application claims benefit of Provisional Appln. 63/136,719, filed Jan. 13, 2021, the entire contents of which are hereby incorporated by reference as if fully set forth herein, under 35 U.S.C. § 119(e).

### BACKGROUND

Different patients process administered drugs differently; and for some drugs or for some neonates, or some combination, the variability in delivery of drug concentration for the same amount administered can have a strong bearing on the effectiveness of the drug. As used herein, the term medicament means any material administered to a subject for therapeutic effect, including drugs and other biological agents, such as large molecules, nucleic acids, viruses and bacteria. In general, one can say that, for some medicaments, careful and precise management of administration and delivery is of critical importance. The process of managing administration and delivery of drugs has been called therapeutic drug management (TDM) in the health care industry. Here we use the term TDM to refer to the management of both drugs and other medicaments.

If the medicament is delivered in too low a dose, the efficacy of the treatment may be compromised; while, if administered in too a high a dose deleterious and even dangerous side effects may occur.

Estimates of individualized response to a medicament is often based on non-linear mixed effects (NLME) models fit to population averages with estimates of an individual's deviation from population averages based on certain parameters, called covariates, such as gender and weight.

### SUMMARY

Techniques are provided for more efficiently determining a patient's response deviation from population averages and therefor more efficient and better dosing of medicaments during individualized treatment of a subject, which dosing has increased chance to avoid costly excursions from a target therapeutic range.

Recently, scientific machine learning (SciML) approaches, also known as physics-informed artificial intelligence (AI), knowledge-guided AI, expert-driven AI, or science-guided AI, have been shown to overcome the issues of ML in situations where data is limited but prior mechanistic knowledge exists in some form. Techniques like universal differential equations and physics-informed neural networks incorporate prior knowledge of mechanistic models into the machine learning process in order to improve the predictive power. These techniques have been shown to be able to augment existing mechanistic models to generate extended models with more predictive power. However, they have been limited to general scientific structures, such as ordinary differential equations, and thus have not been directly applicable to the NLME context generally used within the pharmacometrics community. The embodiments described here provide an extension of the SciML approaches to allow for automated generation of predictive NLME models used in the administration of medicaments.

In a first set of embodiments, a method executed automatically on a processor for generating a dosing protocol for an individual subject, includes receiving first data that indicates, for a dose response to a medicament, a non-linear mixed effects (NLME) model of a population with at least one distribution parameter characterizing variations in the population based on an observable property of individuals within the population. At least one of a structural model or a dynamical model of the NLME model is based on training weights of a universal approximator on a least a subset of the population. The method also includes evaluating, for a candidate dose regimen, an expected response by a subject based on the NLME model and one or more properties of the subject. The method further includes, when the expected response is therapeutic, causing the candidate dose regime of the medicament to be administered to the subject.

In some embodiments of the first set, the universal approximator is a neural network. In some embodiments of the first set, said training weights of the universal approximator is confined to training fixed effect weights of the universal approximator. In some embodiments of the first set, said training weights of the universal approximator includes training random effect weights of the universal approximator to represent deviations of an individual from other individuals with the same observable property. In some embodiments of the first set, the structural model or the dynamical model of the NLME model based on said training fixed-effect weights is implemented as a linear or non-linear model derived from the fixed-effect weights of the universal approximator.

In other sets of embodiments, a computer-readable medium, an apparatus or a system is configured to perform one or more steps of one or more of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example pharmacokinetic and pharmacodynamics system model, used according to an embodiment;

FIG. 2A is a block diagram that illustrates an example neural network 200 for illustration;

FIG. 2B is a plot that illustrates example activation functions used to combine inputs at any node of a neural network;

FIG. 3 is a flow diagram that illustrates an example method for objectively and automatically determining dosing regimen, according to an embodiment;

FIG. 4A through FIG. 4D are plots that illustrate example good fits to simulated data of drug concentration time series by a universal approximator embedded for a portion of a structural model of a NLME model (an example UENLME model) in four different individuals, respectively, according to an example embodiment;

FIG. 4E is a plot that illustrates example good fits to simulated data of drug concentration by the UENLME model for all times and all individuals in a test set, according to an embodiment;

FIG. 5A through FIG. 5E are plots that illustrate example good fits to simulated data of drug concentration in a first truth model by a UENLME model trained on a training set when the trained UENLME model operates on a different test set, according to an embodiment;

FIG. 6A through FIG. 6E are plots that illustrate example good fits similar to FIG. 5A through FIG. 5E but for a second truth model, according to an embodiment;

FIG. 7A through FIG. 7E are plots that illustrate example good fits similar to FIG. 5A through FIG. 5E but for a third truth model, according to an embodiment;

FIG. 8A through FIG. 8E are plots that illustrate example good fits similar to FIG. 5A through FIG. 5E but for a fourth truth model, according to an embodiment;

FIG. 9A and FIG. 9B are plots that compare the actual contributions of 100 covariates in vector Z for a structural model g, to the estimated contributions of those covariates using a symbolic regression derived from the UENLME model, for two dynamic parameters p1 and p2, respectively, according to an embodiment;

FIG. 10A through FIG. 10E are plots that illustrate example good fits similar to FIG. 5A through FIG. 5E but for universal approximator spanning both a structural function g and a dynamical function f, according to an embodiment;

FIG. 11A through FIG. 11E are plots that illustrate example good fits similar to FIG. 5A through FIG. 5E but for a different dosing regimen than in a training set, according to an embodiment;

FIG. 12 is a block diagram that illustrates a computer system upon which an embodiment of the invention may be implemented; and,

FIG. 13 illustrates a chip set upon which an embodiment of the invention may be implemented.

### DETAILED DESCRIPTION

A method and apparatus are described for determining, during treatment of a subject, individualized protocols for administering medicaments, which have increased chance to be maintained in the subject within a therapeutic range. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

In various embodiments, a corresponding precision therapeutics toolset would be directly applicable to all medicaments that benefit from TDM. Prediction models other than those for precision dosing can be incorporated, e.g., to assess appropriateness of palliative chemotherapy in patients with metastatic cancer with dismal prognosis by predicting survival; to use appropriate anti-diabetic therapy in patients with type 2 diabetes of varied pathophysiology; or to predict cardiovascular event risk in a given time period.

### 1. Overview

Biologically and pharmacologically plausible systems models such as Pharmacokinetic (PK) and Pharmacodynamic (PD) (PK/PD) and Physiologically-Based Pharmacokinetic (PBPK) models are tailored for concentration levels of a dose in individual subjects. The systems models for precision therapeutics are highly non-linear and require multiple levels of uncertainty and variation, leading to the use of non-linear mixed effects (NLME) models. NLME for statistical inference is demonstrated in a large literature of applied statistics. Moreover, the form of NLME models limits the types of usable patient data to those that are easily provided quantities such as age, sex, and weight. The systems models proposed here with NLME may be used in conjunction with modern machine learning algorithms such as deep (convolutional) neural networks, density-based clustering on diffusion mapped data, or gradient-boosted regression trees to determine values of model parameters of various categories. This pushes well beyond modeling for the averages to a space filled with complex multi-dimensional healthcare information with both fixed and random effects models. As used herein a random effect is one that has an average value of zero. A fixed effect has a non-zero average value. Such modeling attempts to capture a therapeutically significant subset of the underlying complex pharmacological mechanisms that can be used to individualize therapeutics one patient at a time. As used herein, a subject is a human or animal patient or healthy recruit or surrogate or a simulation of same.

### 1.1 Non-Linear Mixed Effects (NLME) Models

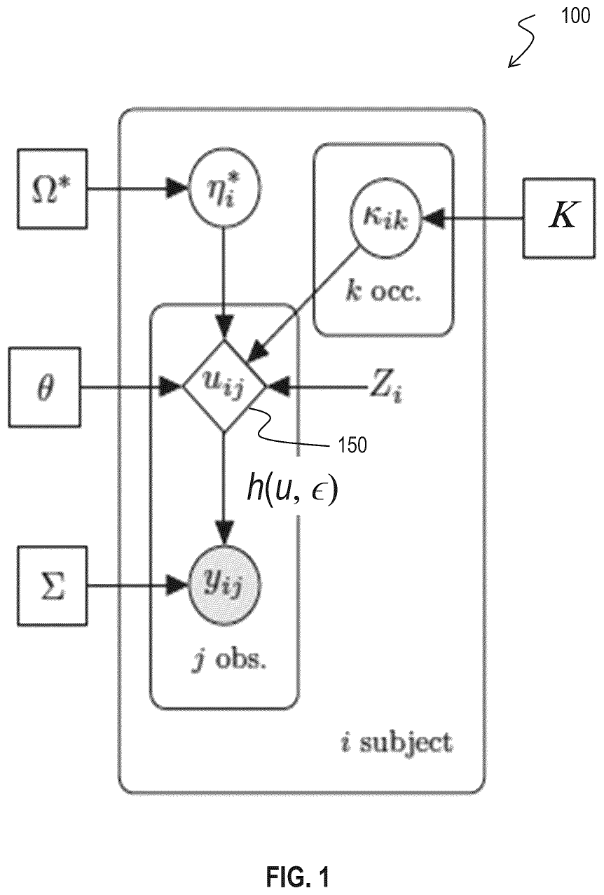

FIG. 1 is a block diagram that illustrates an example pharmacokinetic and pharmacodynamics system model 100, used according to an embodiment. In FIG. 1, the rectangle nodes denote variable parameters, circles denote random quantities which are either latent (unfilled) or observed (filled), diamonds are deterministic given the inputs, and nodes without a border are constant. The system model 100 describes the evolution of one or more observable medicament levels yᵢⱼ for individual i at observation time elements j. Such a system is defined by three components: random effects; dynamics; and observations.

An individual's random effect, represented by the symbol ηᵢ, captures the inter-individual variability (IIV) for individual i within a population of subjects and represents deviations of an individual from other individuals with the same observable property, i.e., with the same values of covariates. The variable ηᵢ, represents one or more random effects for individual i and thus, in general, is a vector. The distribution of this effect in the whole population is parametrized by Ω. Sometimes, one captures other individual random effects, such as the inter-occasion variability (IOV) effect denoted by [symbol] at each occasion k for individual i, e.g., when an individual patient visits a hospital on multiple occasions for a tissue sample to determine medicament levels (such as a blood draw to determine drug concentration value in the blood at those times). [symbol] represents one or more inter-occasion random effects for individual i and thus, in general, is a vector. The distribution of the inter-occasion effect over the whole population is denoted by K The most common choice for random effect components is some sort of multivariate normal distribution N with zero mean of the form ηᵢ=N(0, Ω) and [symbol] =N(0, K).

As used herein, a distribution parameter characterizes variations in the population regardless of origin, e.g., variation among individuals, such as those based on covariates, inter-event variability for an individual, distribution around a population mean of pooled data, and uncertainty in measurements, among others. The symbol θ represents the fixed effects, e.g., the population mean for various dynamical properties, and the deviation from the population mean dynamical properties based on an individual's value Zi for one or more covariates. The symbol ηᵢ represents deviations of an individual from other individuals with the same observable property or properties, i.e., covariate values Zi. For example, after doing a likelihood estimation in something like a Bayesian sense, one learns a distribution for ηᵢ in terms of Ω, but one also might get a distribution for the θ terms. The maximum likelihood estimation (approximately) returns the maximum of this distribution; but, Bayesian fitting methods don't just give one point back but instead a point with uncertainty (similar to an estimate with error bars). So one can have uncertainty in the θ terms by incorporating those error bars. These errors can come from many sources: measurement error in the data, numerical error in the nonlinear routines, etc. Also, in some embodiments, one does not do individual-specific estimations, but still wants to do optimal dosing under the uncertainty with respect to the estimated error in the θ fits.

Dynamics control the time evolution of the system. A dynamic parameter pᵢ represents the dynamics of the model that unfold at a sequence of time points tᵢⱼ for individual i and time element j. The dynamical parameters pᵢ represents one or more effects for individual i and thus, in general, is a vector. A structural model g collates various effects to produce a predicted value for the dynamical parameters pᵢ based on one or more nonrandom population parameters in a vector θ, the one or more random effects η_{I} for individuals in that population including any inter-occurrence variations, and zero or more subject-specific values in vector Zᵢ for covariates variables. Co-variate values Zᵢ is a vector of values for zero or more observable characteristics of individual i, which are known to vary with an observable property of interest yᵢⱼ that is being modeled. In some embodiments, there are no random effects represented by ηᵢ, like in first dose estimation. The general relationship with random effects is given in Equation 1a.

pᵢ=g(θ,Zᵢ,ηᵢ) (1a)

The vector variable uᵢ(t) represents one or more latent properties (such a patient i's internal drug concentration and drug responses) that evolve according to the dynamical parameters. In some embodiments, these dynamics are provided as the solution to a system of differential equations. In other embodiments, the dynamics are modeled in other ways, such as using continuous and discrete Markov chains, or stochastic, delay, hybrid, and differential-algebraic variants of the differential equations. The most common choice is the ordinary differential equation (ODE) approach, which describes the solution uᵢ(t) based on an initial value and a time derivative model f as given in Equation 1b.

uᵢ′=f(t;pᵢ;uᵢ) (1b)

After the dynamical model, an observation model is applied, which relates uᵢ(tⱼ) to the observational data series yᵢⱼ by accounting for measurement errors ϵⱼ. The general form has an observational error structure of a general function h, given in Equation 1c.

yᵢⱼ=h(uᵢ(tⱼ);ϵⱼ) (1c)

In various embodiments, the functional forms for g in Equation 1a, or the form of differential equations f in Equation 1b, or some combination, is replaced in module 150 by a universal approximator, such as a neural network. In simple terms, a universal approximator represents a relationship between values in two spaces, an input space and an output space, as a set of weights operating in a feedforward direction. Stated another way, a universal approximator is a variable-sized parameterization which can approximate any possible function of a chosen target function spac. A Fourier series is a universal approximator of continuous periodic functions on domain [0,2π] with outputs between [−1,1] e. Also, in simple terms, a neural network is one type of universal approximator that emulates neurons in biological systems and uses nodes to represent the input space and the output space, and one or more intermediate layers of nodes in the feedforward direction with switch-on and saturation effects. For example, a neural network with sigmoidal activations on all layers is a universal approximator for the space of continuous functions with outputs between 0 and 1.

In PK compartment models, a subset of NLME models, drug concentrations (C) in the compartments are the dynamic variables u, and are equal to the amounts (A) divided by volumes (V). For two compartments, C1=A1/V1 and C2=A2/V2. Drug concentration in the central compartment (C1) is equal to the concentration in the plasma (CP). Clearance (CL, in units of liters, L, per hour) is often used instead of the fractional rate constants kₐ (in units of per hour, not to be confused with occurrence k, described above and in FIG. 1). In pharmacokinetics, the distribution volume is given in volume units (L), and rate constants can be represented as the ratio of clearance and distribution volume, e.g., kₐ=CL/V. In the case of oral administration of the drug, at time t=0 the amount of drug in the central and peripheral compartments is zero (A1(0)=A2(0)=0), and the initial amount in gastrointestinal tract (effective dose) is: AGI(0)=D×S×F, where D is the administered dose of the drug, S is the salt factor (fraction of administered dose that is made up of pure drug), and F is the bioavailability factor (fraction of dose that reaches the systemic circulation). Other parameters often used in PK models include D=dose, τ=dosing interval, CL=clearance, Vd=volume of distribution, kₑ=elimination rate constant, kₐ=absorption rate constant, F=fraction absorbed (bioavailability), K₀=infusion rate, T=duration of infusion, and C=plasma concentration. In such embodiments, the term Depot refers to the volume into which the drug is originally introduced (e.g., the gastrointestinal tract or the blood stream) and Central refers to the volume in the target area.] The values of such parameters are determined for both the population and for deviation based on values of the covariates, in various levels of approximation using one or more universal approximators.

The simplest individual model is the one-compartment model for which the dynamic model f is defined by the ODE in Equations 2a and 2b.

$$\frac{d [ Depot ]}{dt} = - {k}_{a} [ Depot ] \qquad ( 2 a )$$

$$\frac{d [ Central ]}{dt} = {k}_{a} [ Depot ] - \frac{CL}{V} [ Central ] \qquad ( 2 b )$$

For this model the dynamic parameters pᵢ are listed the vector of Equation 2c

$${p}_{i} = [ \begin{matrix}{k}_{a} \\ CL \\ V\end{matrix} ] \qquad ( 2 c )$$

In a typical structural model g, the covariate values Zᵢ for gender (sex) are 0 for male and 1 for female and weight (wt) are in kilograms as listed in the vector of Equation 2d.

$${Z}_{i} = [ \begin{matrix}{wt}_{i} \\ {sex}_{i}\end{matrix} ] \qquad ( 2 d )$$

Typical random effects among individuals and instances are listed in the vector of Equation 2e.

$${η}_{i} = [ \begin{matrix}{η}_{1} \\ {η}_{2} \\ {η}_{3} \\ {κ}_{k}\end{matrix} ] \qquad ( 2 e )$$

And there are typically four population level values in θ, listed in the vector of Equation 2f.

$$θ = [ \begin{matrix}{θ}_{1} \\ {θ}_{2} \\ {θ}_{3} \\ {θ}_{4}\end{matrix} ] \qquad ( 2 f )$$

And a typical structural model g to generate the values of the 3 parameters pᵢ in Equation 2c is given by the 3 expressions in Equation 2g, respectively (leaving the individual subscript off within each expression for simplicity).

$$g ( θ , {Z}_{i} , {η}_{i} ) = [ \begin{matrix}{θ}_{1} {e}^{{η}_{3} {κ}_{k}} \\ {{θ}_{2} ( \frac{wt}{70} )}^{0.75} {θ}_{4}^{sex} {e}^{{η}_{1}} \\ {θ}_{3} {e}^{{η}_{2}}\end{matrix} ] \qquad ( 2 g )$$

For typical predictive precision dosing, one normally assumes that the average random effect should be zero. Thus, given covariate values Zᵢ of a new patient, one can utilize the population mean effects θ in function g to predict the patient's dynamical parameters pᵢ.

For training and for individualized administration of a medicament, the interest is in the inverse problem of learning more about the model given data of real observations, represented by the symbol dᵢⱼ=dᵢ(tᵢⱼ). The desire is to learn one or more of fixed effects θ, and random effects Ω, Π, Σ, (thus effectively the amount of randomness for the random effects, random occasions and random measurements) or more explicitly at least random draws from those last three distributions ηᵢ, [symbol] and ϵᵢ. This inverse problem, summarized as finding the parameters such that ∥d−y∥ is minimized, is known as the estimation problem. This minimization is performed in prior approaches by finding the parameters that maximize the population likelihood or via Maximum A Posteriori (MAP) estimation. Common methods which approximate the population likelihood include: 1) First order conditional estimation 2) Laplace approximations 3) Expectation maximization (sampling-based methods). In addition, more accurate posterior simulation algorithms such as Hamiltonian Monte Carlo, Stochastic Approximation Expectation-Maximization (SAEM), and Approximate Bayesian Computation (ABC) are used for parameter estimation in some embodiments. In prior work machine learning, such as neural networks, are also used to determine the parameters that define the deterministic, dynamic and random elements of the predetermined structural and dynamical models.

In example embodiments, one or more of these parameters might be described or affected by a distribution about a typical value of random values for different individuals.

Electronic health records (EHR) provide a rich dataset for employing machine learning methods. Thus, in various embodiments, numerical solutions for the differential equations of a NLME model are implemented as an activation layer of a neural net architecture. By directly encoding the ODE dynamics and events into the neural net layer via its activation function, one can retain the properties of the modeling-based approach so that the outputted values (the predicted concentrations) are biologically realistic and follow the proposed clinical model. The population parameters and random distribution function are learned during this training, while maintaining the ODE relationships. In some embodiments, this dynamic layer is then embedded in an end to end neural network that has as input values and any measurements of therapeutic effects on an individual; and; has as an output layer the expected dose response and confidence in (probability of) the same. In other embodiments, other methods of determining the model η parameters are used.

The parameters of this system would then be the input nodes of the dynamical layer, which are chained from earlier layers of a neural network as predictions from arbitrary datasets. In this way, the relationship between electronic health records (EHR) records, imaging results, and molecular biological assays (protein assays, SNP arrays, RNA-seq) can be trained and deduced programmatically without requiring a modeler assumption; directly generalizing previous PK/PD modeling approaches to contemporary big data.

Prior uses of nonlinear mixed effects modeling required specifying the mechanistically motivated form of the structural g and dynamical f models. A change in the current approach to step 301 is to mix a predetermined-model-free inference of machine learning by replacing all or part of these models g or f or h or some combination with universal approximators (such as neural networks), and treating the weights of the approximator as fixed or random effects in a nonlinear mixed effects maximum likelihood estimation. During the fitting algorithm a declaration of which parameters are fixed and random effects is made under the control of the user. The fitting algorithm then produces estimates of fixed effects and a random effect per instance in the training set. So effectively there's no difference other than the user of the NLME algorithm has to declare yes/no for each parameter whether it's a fixed or random effect. Thus, in this case, the “training” process is to use a maximum likelihood estimation to determine the fixed effects θ and the unexplained individual deviations that are random effects ηᵢ, and thus determine the weights of the universal approximator which define the g and f models. In this version of step 301, backpropagation is replaced with the gradient calculation of the maximum likelihood process, which internally involves mixing backpropagation of the neural network with the Jacobian and Hessian calculations of maximum likelihood approximations using differentiable programming techniques (see World Wide Web in subdomain dspace of domain mit in org in folder handle subfolder 1721.1 file 137320).

For example, in a Bayesian estimation process, given observations yᵢ of the underlying dynamical variables uᵢ for multiple individuals i, and occurrences k, and given the likelihood of given fixed effects θ, and given the random effects variance Ω, and occurrence variability characterized by K, the population likelihood L is related to the individual likelihoods Lᵢ by Equation 3a.

L(Ω,θ,K,y)=ΠᵢLᵢ(uᵢ,yᵢ) (3a)

Here, Π indicates the product of the individual likelihoods (as distinguished from distribution Π FIG. 1). Lᵢ is the likelihood for individual i which is a chosen functional form. For example, a common choice of Lᵢ is the normal distribution centered around the data with measurement error variance σ, such as given by Equation 3b.

Lᵢ˜N(uᵢ−yᵢ,σ) (3b)

The fixed effect parameters θ, Ω, K and random effect parameters ηᵢ are chosen to maximize the likelihood of the observation, which is equivalent to choosing the parameter values that minimize ∥uᵢ−yᵢ∥. In this Bayesian estimation procedure, it is desired to capture the uncertainty in the parameters θ, Ω, K and ηᵢ instead of performing a deterministic procedure, like maximum likelihood. In the Bayesian approach, the posterior probabilities distributions for θ, Ω, K and ηᵢ are derived based on assumed prior distributions. This is done iteratively starting with an initial distribution, such as a normal distribution.

Common probabilistic programming languages, such as Stan or Turing, can be utilized to determine these posterior distributions using Markov Chain Monte Carlo (MCMC) techniques such as Hamiltonian Monte Carlo. Plots of estimated distributions of NLME parameters computed using the Pumas and other software via HMC against data, showcase how the discrete MCMC chains can be reinterpreted as probability distributions via a kernel density estimate (KDE). In various embodiments, a universal approximator is trained to determine the parameters of θ or Ω or K, or some combination, as fixed effects

### 1.2 Neural Networks

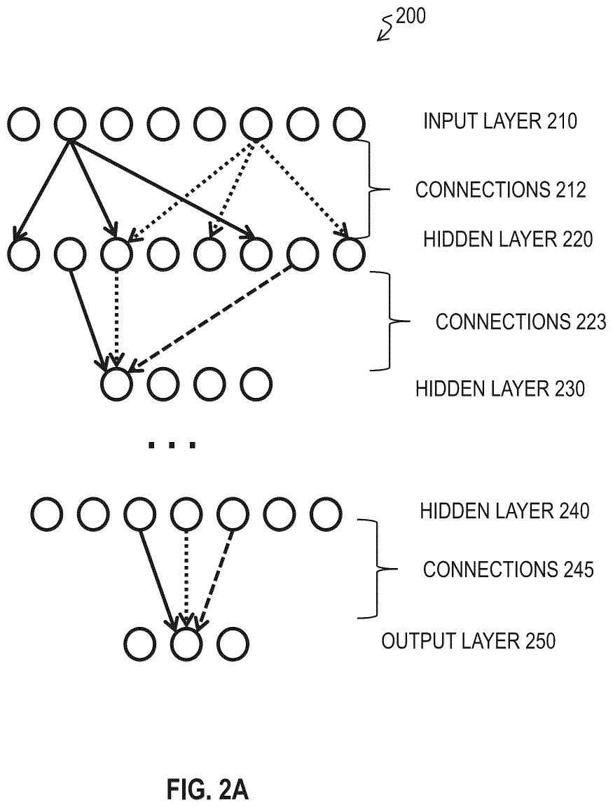

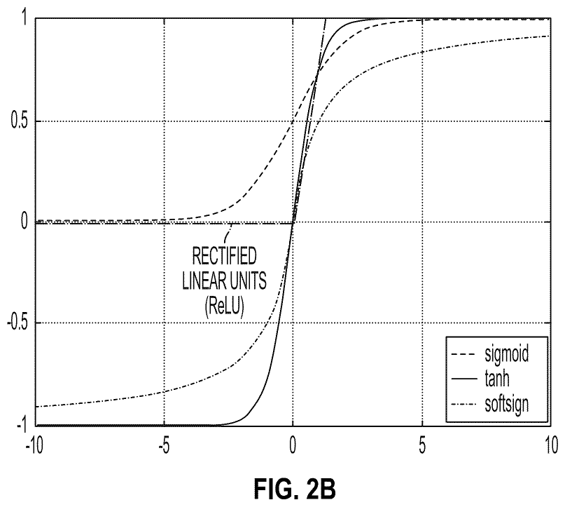

Effective training of universal approximators with the characteristics described above can be achieved using neural networks, widely used in image processing and natural language processing. FIG. 2A is a block diagram that illustrates an example neural network 200 for illustration. A neural network 200 is a computational system, implemented on a general-purpose computer, or field programmable gate array, or some application specific integrated circuit (ASIC), or some neural network development platform, or specific neural network hardware, or some combination. The neural network is made up of an input layer 210 of nodes, at least one hidden layer 220, 230 or 240 of nodes, and an output layer 250 of one or more nodes. Each node is an element, such as a register or memory location, that holds data that indicates a value. The value can be code, binary, integer, floating point or any other means of representing data. Values in nodes in each successive layer after the input layer in the direction toward the output layer is based on the values of one or more nodes in the previous layer. The nodes in one layer that contribute to the next layer are said to be connected to the node in the later layer. Connections 212, 223, 245 are depicted in FIG. 2A as arrows. The values of the connected nodes are combined at the node in the later layer using some activation function with scale and bias (also called weights) that can be different for each connection. Neural networks are so named because they are modeled after the way neuron cells are connected in biological systems. A fully connected neural network has every node at each layer connected to every node at any previous or later layer.

FIG. 2B is a plot that illustrates example activation functions used to combine inputs at any node of a neural network. These activation functions are normalized to have a magnitude of 1 and a bias of zero; but when associated with any connection can have a variable magnitude given by a weight and centered on a different value given by a bias. The values in the output layer 250 depend on the values in the input layer and the activation functions used at each node and the weights and biases associated with each connection that terminates on that node. The sigmoid activation function (dashed trace) has the properties that values much less than the center value do not contribute to the combination (a so called switch off effect) and large values do not contribute more than the maximum value to the combination (a so called saturation effect), both properties frequently observed in natural neurons. The tanh activation function (solid trace) has similar properties but allows both positive and negative contributions. The softsign activation function (short dash-dot trace) is similar to the tanh function but has much more gradual switch and saturation responses. The rectified linear units (ReLU) activation function (long dash-dot trace) simply ignores negative contributions from nodes on the previous layer, but increases linearly with positive contributions from the nodes on the previous layer; thus, ReLU activation exhibits switching but does not exhibit saturation. In some embodiments, the activation function operates on individual connections before a subsequent operation, such as summation or multiplication; in other embodiments, the activation function operates on the sum or product of the values in the connected nodes. In other embodiments, other activation functions are used, such as kernel convolution.

An advantage of neural networks is that they can be trained to produce a desired output from a given input without knowledge of how the desired output is computed. There are various algorithms known in the art to train the neural network on example inputs with known outputs. Typically, the activation function for each node or layer of nodes is predetermined, and the training determines the weights and biases for each connection. A trained network that provides useful results, e.g., with demonstrated good performance for known results, is then used in operation on new input data not used to train or validate the network.

In some neural networks, the activation functions, weights and biases, are shared for an entire layer. This provides the networks with shift and rotation invariant responses. The hidden layers can also consist of convolutional layers, pooling layers, fully connected layers and normalization layers. The convolutional layer has parameters made up of a set of learnable filters (or kernels), which have a small receptive field. In a pooling layer, the activation functions perform a form of non-linear down-sampling, e.g., producing one node with a single value to represent four nodes in a previous layer. There are several non-linear functions to implement pooling among which max pooling is the most common. A normalization layer simply rescales the values in a layer to lie between a predetermined minimum value and maximum value, e.g., 0 and 1, respectively.

### 1.3 Method for Using Models in Administering Medicaments

Once the model parameters and their uncertainties are determined, the model is used to determine the dosing regimen for an individual. The dosing regimen specifies how much, where and when to administer the medicament to the subject. As used herein a “dose” refers to an amount of a medication that is administered to a position inside the subject at a particular time, rather than the resulting concentration level in a target tissue of the subject. Because of the random components describing variations among individuals and variations in results between events of a single individual, any particular dose can lead to a range of possible outcomes in terms of actual observed medicament concentrations. An understanding of that range of possible results should inform the decision on the original dose. In many prior approaches, the expected result is expressed in terms of the probability of the actual concentration occurring in a therapeutic range. If that probability is high enough for a particular dose, then that dose is considered acceptable.

It is noted here that, of all acceptable doses, a subset may be preferable because the probability of deleterious deviations from the therapeutic range are smaller than for other administered doses in the acceptable range. Therefore, in various embodiments, a cost function is defined that includes a measure of weighted deviations from the therapeutic range; and the dose determined is based on reducing or minimizing the weighted deviations. This formulation reduces or minimizes a cost, where the cost is based on the probability of high excursions of the subject's drug response from known safety windows. While this cost function can result in high values, it is a better indication of a true cost to the subject; and, thus; is more useful for optimizing dose (compared to minimizing the distance from some optimal response curve, which does not capture the fact that any solution within the safety window is “good”). Thus, the quantity of interest in some embodiments, is the probability of rare but large excursions.

In some embodiments, this cost is evaluated based on using a Koopman adjoint approach to determine the distribution of initial conditions, e.g., dose, which approach is several times more efficient than a naïve Monte Carlo approach to determining that distribution. In some embodiments, increased efficiency is achieved by employing graphical processing unit (GPU) accelerated data-parallel numerical solutions to ordinary differential equations (ODEs), in addition to, or instead of, the Koopman adjoint approach.

It is assumed that Bayesian estimation or prior knowledge or other technique, such as maximum likelihood, has given sufficient estimates of posterior distributions for θ and ηᵢ and the following question is to be answered: what is an optimal or advantageous dosage regimen Dᵢ for patient i? The rest of this section is focused on this single patient i; and, thus the subscript i will be dropped for this section.

Cost functions are advantageously based on clinical trials used to establish safety guidelines known as the therapeutic window. For example, for a given drug the area under the curve (the total concentration or AUC) of the drug concentration in the target tissue or other dynamic variable u (or measurement y dependent on u) is a common quantity in which therapeutic windows are written. Given how every individual metabolizes at given rates, the goal is to optimize the probability that the dose will be in the therapeutic window with respect to the uncertainty in the patient-specific effects. Any cost function may be used. In some embodiments, the cost function not only accounts for “goodness” in giving preferential weight (low cost) to results in the therapeutic window but also allows for cost of “badness,” such as avoiding certain ranges by assigning high cost to those ranges or increasing cost for distance from the therapeutic window. Using distance removed from the therapeutic window encapsulates the idea that being close to the therapeutic window might be sufficient while being far away incurs a greater risk on the patient.

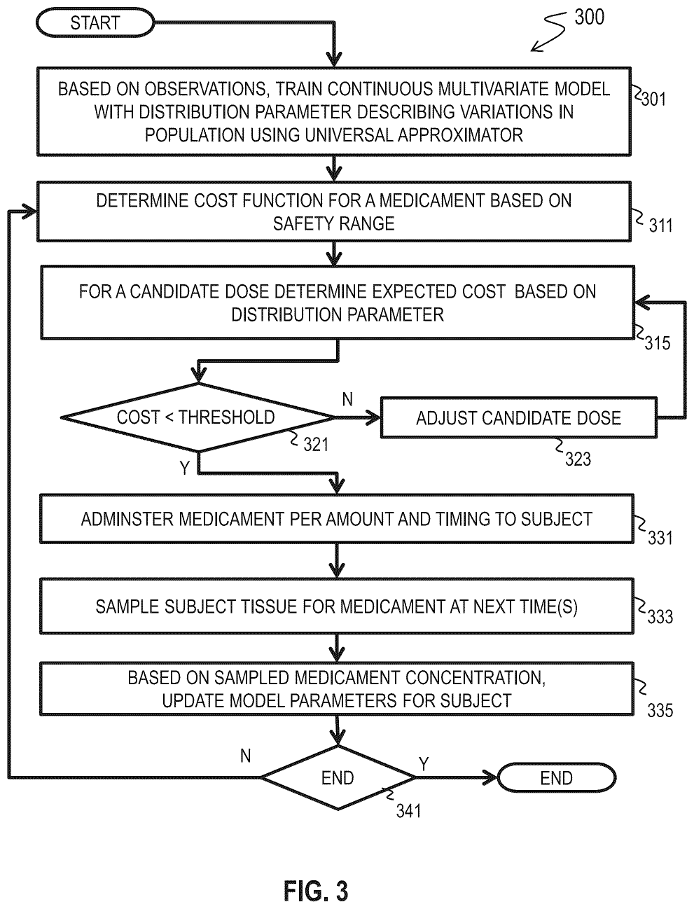

FIG. 3 is flow diagram that illustrates an example method for determining a dose that will minimize a cost, according to an embodiment. Although steps are depicted in FIG. 3 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 301, a NLME model is developed with appropriate random and nonrandom parameters; and, the model parameters are learned based, for example, on historical data in the EHR by training a universal estimator representing the structural model or the dynamical model or both. The training continues until a certain stop condition is achieved, such as a number of iterations, or a maximum or percentile difference between model output and measured values is less than some tolerance, or a difference in results between successive iterations is less than some target tolerance, or the results begin to diverge, or other known measure, or some combination. More details on a universal approximator embedded NLME and its training in provided in the next section on example embodiments.

As a result of the training, the model is a continuous multivariate model that requires no classification or binning of the subject be performed, as is required in the application of the nomograms of the prior art. The model includes, in θ, at least one parameter based on population average and at least one parameter describing dependence on individual covariate values Z, and at least one parameter (e.g., Ω) describing random deviations among individuals as well as at least one portion describing dynamics (e.g., using ordinary differential equations or universal approximator, or some combination, describing changes over time). As a result of step 301, a continuous multivariate model is received on a processor as first data that indicates, for dose response to a medicament, with at least one distribution parameter. This is a non-linear mixed effects (NLME) model of a population with at least one distribution parameter characterizing variations in the population based on an observable property of individuals within the population wherein at least one of a structural model or a dynamical model of the NLME model is based on training fixed-effect weights of a universal approximator on a least a subset of the population.

In step 311 a cost function is determined based on a safety range, such as a therapeutic range or a therapeutic index or some combination. In some embodiments, the cost function includes a weighted function of distance from the therapeutic range. For example, there is no cost for a result within the therapeutic range, and an increasing cost with increasing distance above the therapeutic range. That can be linear or have any shape function with distance. In some embodiments, an especially toxic range, e.g., as determined based on the therapeutic index, is given a higher weight that is not necessarily proportional to distance from the therapeutic range. In embodiments implemented automatically on a processor, step 311 includes: receiving second data that indicates a therapeutic range of values for the dose response; and, receiving third data that indicates a cost function based on distance of a dose response from the therapeutic range.

In step 315, for a candidate dose, the expected cost of that candidate dose is determined based on the at least on parameter of random deviations (e.g., Ω for each of one or more model parameters). In some embodiments the Koopman transform of the cost function using multidimensional quadrature methods is also determined. In some of these embodiment the evaluation is performed, e.g., based on known graphical processing unit acceleration techniques.

In step 321, it is determined whether the cost is less than some target threshold cost, such as a cost found to associated with good outcomes. If not, control passes to step 323 to adjust the candidate dose, e.g., by reducing the candidate dose an incremental amount or percentage. Control then passes back to step 315 to recompute the cost. If the evaluated cost is less than the threshold, then control passes to step 331.

In step 331 the medicament is administered to the subject per the candidate dose. Thus, when the expected cost is less than a threshold cost, the candidate dose of the medicament is caused to be administered to a subject.

In step 333, a sample is taken from the subject to determine therapeutic effect (e.g., concentration of the medicament in the tissue of the sample) at the zero or more measurement times determined during step 325. This step is performed in a timely way to correct any deficiencies in administering the medicament before harmful effects accrue to the subject, such as ineffective or toxic results. Thus, step 327 includes sampling the subject at the set of one or more times to obtain corresponding values of the corresponding measures of the therapeutic effect.

In step 335 the model determined in step 305 is updated, if at all, based on the measurements taken in step 333. For example, if the medicament concentration value is less than or greater than the therapeutic range, it is determined that the individual has a response that deviates substantively from the population average or covariate-deduced values. Any method may be used to update the model parameters to fit the observed response of the individual, such as new training of the neural network. In some embodiments, the population model is updated based on the one observation of the individual being treated in proportion to the number of different subjects that previously contributed to the model. In some embodiments, in addition to or instead of updating the population model parameter values, special subject-specific model parameter values are generated.

The method 300 thus determines, during treatment of a subject, individualized protocols for administering medicaments, which have increased chance to be maintained in the subject within a therapeutic range. The extra formalism is useful because of the tremendous performance and feature benefits.

As personalized precision dosing becomes more commonplace, efficient and robust methods for optimizing dosing with respect to probabilistic outcomes will become more central to pharmacometric practice. As personalized computations become pervasive, more accurate models will be required, and the computations will need to migrate to the patient, living on embedded or mobile devices with little compute power.

### 2. Example Embodiments

In various embodiments, the method 300 is configured to include universal approximators in step 301. As used herein, a universal approximator U(x;ϕ) is a parameterized function such that, for any nonlinear function φ: Rⁿ→R^{m} that satisfies theorems of universal approximators and for any tolerance E>0, there exists parameters ϕ such that Equation 4 is true

∥U(x,ϕ)−φ(x))∥<E (4)

for all x in the domain. The most common universal approximators are deep neural networks, where with large enough layers or enough layers, a neural network is a universal approximator. However, other common universal approximators include Fourier series, polynomial expansions, Chebyshev series, recurrent neural networks, convolutional neural networks, shallow networks, and more which have this mathematical property. These universal approximators can be computed using parallelism within the function U, across the multiple universal approximators of the NENLME model, across patients, and other splits which accelerate the computation. The universal approximator's calculations can be accelerated using hardware compute accelerators such as GPUs and TPUs, as is commonly used with neural networks.

In various embodiments, one or more unknown portions of an NLME model are replaced with universal approximators in order to automatically learn the structure from data. This is done by defining either part or all of the components f, g, and h with universal approximators whose weights or inputs are functions of the random and or fixed effects. The weights of the approximators themselves are then treated as either random or fixed effects (or some combination thereof) in order to be fit using standard NLME fitting routines as described in the later section. After fitting the weights of the universal approximators, an optional analysis step can be performed known as a symbolic regression (discussed in file 2001.04385 in folder abs on domain arxiv with extension org on the World Wide Web), which transforms the neural networks into predicted mechanistic structures to arrive at a form of the model with no universal approximators. It was found to be advantageous for the universal approximator weights to represent the fixed effects and to select the training options accordingly.

### 2.1 Universal Approximators in Structural Model g

Where the structural model is unknown, the model can be replaced, in full or in part, by a universal approximator that can be trained directly from the data. For example, when one knows that the above described one-compartment model well-described the internal drug dynamics, and when one has an expansive set of covariates Zᵢ for a large patient population, one could replace the full structural model g with a neural network as given in Equation 5a

pᵢ=U(θ,Zᵢ,ηᵢ,ϕ) (5a).

Because U is a universal approximator, given a large enough neural network, there exists structural parameters ϕ (also known as the universal approximator's fixed weights, for example the fixed weights of the neural network when U is a neural network) such that U well-approximates the behavior of the true underlying g. Thus estimating the universal approximator's weights ϕ serves as a method for estimating the entire function g. This extends the previous NLME estimation problem, which was concerned about point estimates θ and ηᵢ to the estimation of the structural functions. The weights ϕ can then be treated as population-wide averages and covariate dependencies (fixed effects), per-subject variation (random effects), or some combination thereof and fit using the NLME estimation methods. As stated above, it was found to be advantageous for the universal approximator to be trained for the fixed effects and thus give the population wide parameter values for population-wide averages and covariate dependencies. Optionally, the structural form of this previously unknown term could then be recovered using symbolic regression techniques such as (Implicit) Sparse Identification of Nonlinear Dynamics (SINDy), evolutionary and genetic algorithm approaches, OccomNets, and other equation discovery approaches, by sampling a dataset of (input, output) pairs on the universal approximator and performing the equation discovery on the dataset, or by performing the equation discovery algorithm directly on the learned functional form.

In some embodiments, aspects of these inputs are ignored. For example, the universal approximator for the dynamics can be trained without inputting the random effects to learn a dynamical model which is not perturbed for individual patients deviations from the covariate dependencies observed in the population. Additionally, it is noted that g could also be a function of pᵢ itself, either explicitly having parts of the vector functions of prior calculated portions or fully implicitly to impose constraint equations.

In another embodiment, the structural model g is partially mechanistic and partially described by a universal approximator. For example, in the prior NLME modeling example, the structural function g contained a complex interaction between the covariates of weight and sex in order to determine the clearance rate as given by the middle expression in Equation 2g. That required that such a functional form was known by the modeler in advance. Instead, in some embodiments, this structural assumption is removed by replacing the functional form with a universal approximator, leading to the form (called a universal approximator-embedded form, or for brevity neural-embedded form, though other non neural net universal approximators may be used) as given in Equation 5b.

$$g ( θ , {Z}_{i} , {η}_{i} ) = [ \begin{matrix}{θ}_{1} {e}^{{η}_{3} {κ}_{k}} \\ {θ}_{2} {e}^{{η}_{1}} U ( wt , sex , ϕ ) \\ {θ}_{3} {e}^{{η}_{2}}\end{matrix} ] \qquad ( 5 b )$$

Because Equation 5 no longer requires knowing a prior model for determining clearance from the covariates, one could additionally expand the covariates parameters in θ beyond wt and sex, and use the additional covariates as inputs for the universal approximator term. For example, the covariates Zᵢ vector can be expanded to include data from real-world images, omics data (genomics, transcriptomics, proteomics, metabolomics, etc.), EHR data, digital health data collected from wearable technologies, and other aspects of patient's information and biology which can be obtained. This form is given in Equation 5c.

$$g ( θ , {Z}_{i} , {η}_{i} ) = [ \begin{matrix}{θ}_{1} {e}^{{η}_{3} {κ}_{k}} \\ {θ}_{2} {e}^{{η}_{1}} U ( {Z}_{i} , ϕ ) \\ {θ}_{3} {e}^{{η}_{2}}\end{matrix} ] \qquad ( 5 c )$$

Thus, strategic use of universal approximators allows for the more complete exploitation of domain knowledge, compared to typical methods, and therefore allows for the predictions of the neural networks to be constrained and minimized while standing in the physical interpretation context of the traditional NLME framework.

### 2.2 Universal Approximators in Dynamical Model f

In another example embodiment, the dynamical model f is replaced in full or in part by a universal approximator that can be trained directly from data. For example, the structural model in known in full or in part (as described in in 2.1), the dynamical model f can be written as a function of a universal approximator in full or in part.

In the full form, the dynamical model f would be the given by Equation 6a.

u′=U(u,pᵢ,θ,Zᵢ,ηᵢ,θ) (6a)

That is, the universal approximator is a function of the state of the dynamical model u, the output of the structural model pᵢ the fixed effects θ including the dependencies among covariate parameters, the random effects ηᵢ, and the universal approximator's weights ϕ. The weights ϕ can then be treated as population-wide including per subject covariate effects, (fixed effects), unexplained variation among individuals (random effects), or some combination thereof and fit using the NLME estimation methods. When the NLME fitting technique is the naïve pooled method, this is an extension to the neural ordinary differential equation which incorporates covariate and fixed effects information, though other NLME fitting techniques can be used which further improve patient-specific fitting. Aspects of these inputs can be ignored, for example, the universal approximator for the dynamics can be trained without inputting the covariates to learn a dynamical model which is not perturbed according to said data. The state vector u may live in an enlarged dynamical space, e.g., a subset of u may describe true physical states while augmented values describe latent state variables.

It is noted that, for simplicity, the described form was an ordinary differential equation (ODE), though an extension to stochastic differentiation equations (SDEs) arises by defining both the drift and diffusion equations of an SDE in this manner by one or two universal approximators. Similar extensions to delay differential equations (DDEs), differential-algebraic equations (DAEs), and other dynamical models such as Gillespie models (Poisson processes) can similarly be done by replacing functional forms by universal approximators. Additionally, it is noted that f could also be a function of u′ itself, either explicitly having parts of the vector functions of prior calculated portions or fully implicitly to impose constraint equations.

In another embodiment the dynamical model is partially described by some prior known or assumed model which is then augmented by a universal approximator. If the ODE form of Equations 2a and 2b was not completely known, or known to only be an insufficient approximation of the underlying dynamics, the model could be extended with universal approximators. For example, an assumption of structural uncertainty in both of the differential equations would result in an universal approximator embedded model where the dynamical model f can be written as given in Equations 6b and 6c.

$$\frac{d [ Depot ]}{dt} = - {k}_{a} [ Depot ] + {U}_{1} ( [ Depot , Central ] , {p}_{i} , θ , {Z}_{i} , {η}_{i} , {ϕ}_{1} ) \qquad ( 6 b )$$

$$\frac{d [ Central ]}{dt} = {k}_{a} [ Depot ] - \frac{CL}{V} [ Central ] + {U}_{2} ( [ Depot , Central ] , {p}_{i} , θ , {Z}_{i} , {η}_{i} , {ϕ}_{2} ) \qquad ( 6 c )$$

Not only does this formulation allow the covariates to be expanded but also the state variables u to included other states, such as additional latent states, e.g., distinguished from Depot and Central by the term Augmented (A), as expressed in Equations 6d to 6f.

$$\frac{d [ Depot ]}{dt} = - {k}_{a} [ Depot ] + {U}_{1} ( [ Depot , Central , A ] , {p}_{i} , θ , {Z}_{i} , {η}_{i} , {ϕ}_{1} ) \qquad ( 6 d )$$

$$\frac{d [ Central ]}{dt} = {k}_{a} [ Depot ] - \frac{CL}{V} [ Central ] + {U}_{2} ( [ Depot , Central , A ] , {p}_{i} , θ , {Z}_{i} , {η}_{i} , {ϕ}_{2} ) \qquad ( 6 e )$$

$$\frac{d [ Depot ]}{dt} = {U}_{3} ( [ Depot , Central , A ] , {p}_{i} , θ , {Z}_{i} , {η}_{i} , {ϕ}_{3} ) \qquad ( 6 f )$$

While different universal approximators are show to have different weights ϕ_{I}, ϕ2, etc., in practice, in some embodiments neural networks have shared layers which is a form of compression. In this context, ϕ is the concatenation of the weights ϕ_{I}, ϕ2, etc. of all universal approximators in a given NLME model.

The universal structural form of Equations 6b and 6c or of Equations 6d through 6f, can then be fit as described in the following section to obtain the approximator's weights which best fit against the data, giving a predictive dynamical model in an NLME model. The universal approximator can then optionally be symbolically regressed using the previously mentioned techniques to recover a prediction of a mechanistic model form.

It is noted that, when describing multiple universal approximators in the model, the above mathematical forms need not correspond to a split in the functions computationally. For example, in the prior universal approximator embedded ordinary differential equation f, it may be computed using a universal approximator U which is a neural network with 3 output nodes which correspond respectively to the elements of vector [U₁, U₂, U₃].

### 2.3 Universal Approximators in Observational Model h

In another example embodiment, the observational model h is replaced in full or in part by a universal approximator that can be trained directly from data. For example, the structural model g and dynamical model h are known in full or in part (as described above), the observational model h can be written as a function of a universal approximator as given in Equation 7a.

yᵢⱼ=U(uᵢ(tⱼ),ϵᵢ,ϕ) (7a)

Equation 7a relates the dynamical predictions uᵢ(tⱼ) to the observational data series yᵢⱼ through the measurement errors ϵᵢ and the universal approximator's weights ϕ. The universal approximator could also be a function of the yᵢⱼ itself, implicitly or explicitly of other yᵢⱼ. The weights ϕ can then be treated as population-wide (fixed effects), per-subject (random effects), or some combination thereof and fit using the NLME estimation methods. The universal approximator can then optionally be symbolically regressed using the previously mentioned techniques to recover a prediction of a mechanistic model form.

In another embodiment, different forms of the universal approximator is used to perform different kinds of discovery. For example, the universal approximator in Equation 7a could be a dense neural network to learn optional likelihood functions for the observational variables. In another embodiment, U is chosen as a recurrent model given by Equation 7b.

yᵢⱼ=U(yᵢ₍ⱼ₋₁₎,uᵢ(tⱼ),ϵᵢ,ϕ) (7b)

Then the learned form would be a Markov model on the functional form. This form could be used. for example, to learn links to pharmaco-economics data or models and incorporate real-world evidence data.

In another embodiment, the observational model is partially described by universal approximators to allow for incorporating prior knowledge. For example, in one such embodiment, it is assumed that the observational model is a Markov model that is linear with respect to its updates, for some known or learnable α fit either as a fixed or random effect. This form is given by Equation 7c.

yᵢⱼ=αyᵢ₍ⱼ₋₁₎,+U(uᵢ(tⱼ),ϵᵢ,ϕ) (7c)

### 2.3 Training Universal Approximators in NLME Models

Training universal-approximator-embedded NLME (UENLME) models is performed by allocating the universal approximator weights ϕ for each of the number M of embedded approximators (Uₘ, m=1 to M) to either fixed or random effects. For example, all of the weights of the approximators could be chosen to be fixed effects, or some of the weights could be chosen to be random effects while others are fixed effects, or all can be chosen to be random effects. One this choice is made, the training of the NENLME model turns into a maximum likelihood estimation of the fixed and random effects against data, including from traditional clinical sources but also from big data sources such as nucleic acid sequences, omics (genomics, transcriptomics, proteomics, metabolomics, etc.), EHR, among others. It was determined that it was advantageous to treat all the weights as fixed effects to discern the physical dependence.

The weights can be discovered as in traditional NLME(leaving out the inter-occurrence variability K for simplicity but straightforwardly extendable in the manner described), in which the log-likelihood of the parameters L(θ,Ω) where η is distributed according to a probability distribution parameterized by Ω, is given by Equation,

L(θ,Ω)=Σᵢ log [symbol] p{ϵ(ηᵢ)|Σ}p{ηᵢ|Ω}dηᵢ (8)

where Σ is the prescribed observational error distribution, as defined earlier. This equation can be interpreted as follows: for a given possible random effect ηᵢ, the probability of seeing the measurement errors ϵ gives a likelihood for the parameters. Many NLME estimation techniques can be used, such as the first-order conditional estimation (FOCE) with and without interaction (FOCE and FOCEI), first-order with and without interaction (FO and FOI), the Laplace approximation with and without interaction (Laplace and LaplaceI), direct quadrature approximations of the likelihood, and more which approximate this likelihood to calculate the Maximum Likelihood Estimates (MLE) for θ and Ω. These can give the largest probability of encountering the data, subject to the constraint that the expected value of Ω, E|Ω|, ≈0. A full description of NLME fitting methodologies is provided in the An overview of the math in nonlinear mixed effects models is available on the World Wide Web at subdomain link of domain springer of com in folder article search item 10.1007%2Fs10928-007-9060-6. After this likelihood estimation is performed, the optional symbolic regression can be performed in order to transform the trained universal approximator U into simpler mechanistic forms by either sampling an input/output dataset from the learned approximator or applying symbolic regression techniques directly to the learned operator.

Other estimation techniques, such as Stochastic Approximation of Expectation Maximization (SAEM) generate possible ηᵢ distributions and utilize these distributions to update the predicted θ and Ω from data, iterating until an MLE estimate is reached. Still other estimation techniques, such as Bayesian methods like Markov Chain Monte Carlo (MCMC) and derivatives like Hamiltonian Monte Carlo (HMC) give posterior probability distribution estimates for θ and Ω utilizing prior distributions. This thusly defines a posterior probability on the universal approximator weights ϕ which gives a probabilistic description of the learned structures. Symbolic regression techniques can be optionally performed on this probabilistic setting by sampling probable universal approximator weights ϕ from the posterior distribution and performing symbolic regression on the samples to obtain probability distributions description the potential mechanistic forms.

In some embodiments, the training of these methods, such as FOCE(I), FO(I), Laplace(I), and HMC, involves calculating gradients of the models. This gradient calculation can be done using automatic differentiation, also known as back-propagation in the case of neural networks, to calculate gradients, Jacobians, and Hessians of embedded universal approximators. This automatic differentiation can be done in forward or reverse. This calculation could additionally be done in some embodiments using other methods such as analytical gradients, symbolic gradients, finite difference gradients, and complex-step differentiation. The differentiation of the dynamical model can similarly be done using multiple automatic differentiation, sensitivity analysis, and adjoint approaches as described in references. Automatic differentiation generally increases the speed and accuracy of derivative computations.

### 2.4 Example Performance of Universal Approximators in NLME Models

In all the following examples, the truth model is a known NLME model which is used to generate an underlying data source to measure the effectiveness of the universal approximator-embedded NLME (UENLME) technique. These truth models are expert-generated models which closely match the features and characteristics of clinical trial data where NENLME is being used in practice. The population average prediction is the prediction one arrives at using current NLME fitting techniques assuming Zᵢ=0 and ηᵢ=0, while the DeepNLME prediction is the one arrived at using UENLME with the described deep neural networks as universal approximators as described per section. All of these examples demonstrate that the UENLME technique is able to greatly outperform standard NLME in discovering patient-specific predictive models with non-zero values for covariates values Zᵢ and deviations therefrom η₁; and is subsequently shown to improve the ability to give greatly improved performance in predicting the patient outcome under new dosage regimens. Additionally, these examples showcase how the UENLME technique can be interpreted via symbolic regression to allow practitioners to describe what aspects of the extended data, such as EHR, -omics, and images, were used by the learned NUENLME model to improve the predictions.

In one embodiment, UENLME modeling was applied to 10 covariates considered to be linear prognostic factors to learn a structural model g where the dynamical system f was an assumed known form (one-compartment model). The covariates were weight, gender and 8 other EDR values. Simulated data for the values of the three dynamic parameters pᵢ(kₐ, CL, V) was generated with the linear g model where 100% of the subject variation in these values was explainable by the covariates. This means that Ω was zero for all three parameters. The three θ values were 2.7, 1.0 and 2.8, respectively. Simulated dynamic output data was generated by solving the resulting NLME model including a known observation model h with errors ϵᵢ using Pumas with 200 training subjects. An UENLME model was prepared in which the actual g was replaced by a universal approximator (a neural network) driven functional form given by Equation 9 with m=1, M different dynamical parameters.

log(pᵢ)=log(θₘ)+exp(Uₘ(Zᵢ,ϕ)) (9)

The model of Equation 9 was then trained on the resulting dataset where the weights ϕ were interpreted as fixed effects. The predictions of the UENLME model on subject with new covariates was then calculated.

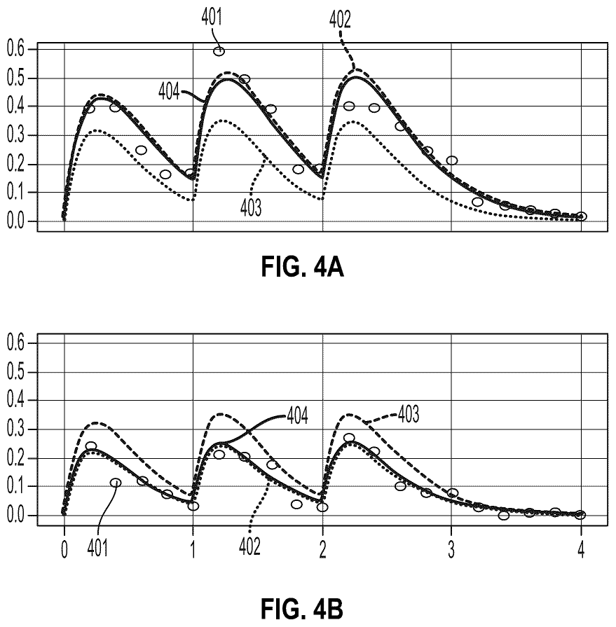

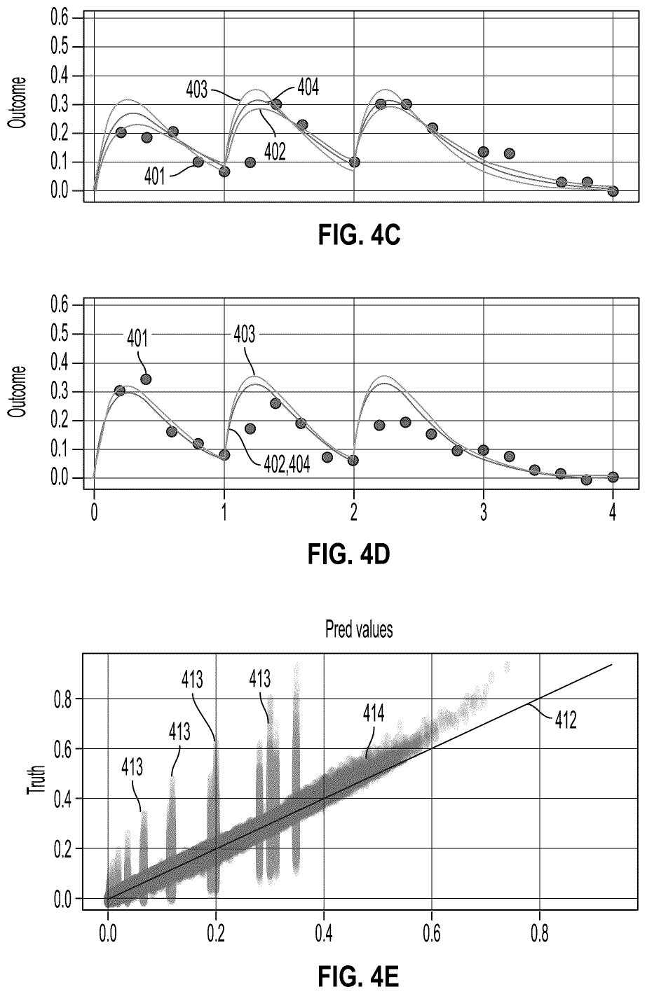

FIG. 4A through FIG. 4D are plots that illustrate example good fits to simulated data of drug concentration time series by a universal approximator embedded for a portion of a structural model of a NLME model in four different individuals, respectively, according to an embodiment. In each plot, the horizontal axis is identical and indicates time from 0 to 4 weeks; and the vertical axis is identical and indicates outcome (normalized drug concentration) on a scale from 0 to 0.6. Data points 401 representing yᵢⱼ are indicated by dots, the true values for uᵢⱼ is represented by trace 402, the population average that does not consider the covariates among individuals is represented by trace 403, and the UENLME model results after training is represented by trace 404. In FIG. 4A through FIG. 4D the difference between the UENLME model's patient-specific outcome 404 is plotted with the true outcome 402 calculated by simulating the original structural g model on a subject (set of covariates) that was not included in the original training data. FIG. 4E is a plot that illustrates example good fits to simulated data of drug concentration by the UENLME model for all times and all individuals in a test set, according to an embodiment. The horizontal axis indicates estimates of normalized drug concentration (uᵢⱼ) from various sources and the vertical axis indicates the correct values of uᵢⱼ. The true values for uᵢⱼ are represented by trace 412. The population average that does not consider the covariates among individuals is represented by points 413 and deviates widely from the true values (e.g., when the true value is 0.2, the population average can be anywhere from about 0.003 to about 0.36) and never reaches the higher values at or above about 0.4). The UENLME model results after training are represented by points 414 that cluster nicely about the trace 412 (e.g., when the true value is 0.2 the UENLME model produces values from about 0.15 to about 0.25 and dose reach the highest values, although somewhat more rapidly than truth).

These plots demonstrate that the UENLME model is able to learn to give patient-specific predictions of internal drug concentrations (pharmacokinetics) and patient drug responses (pharmacodynamics) in a way that sufficiently accurately deviates from the population average 403. This shows that UENLME modeling can be used to do patient-specific optimizations of dosage regimens, improve clinical trial designs, predict the outcomes of noncompartmental analyses (NCA), and learn patient-specific absorption profiles for applications in in-vitro in-vivo correlation (IVIVC) and in-vitro in-vivo relationship (IVIVR) analyses.

In another set of embodiments, UENLME modeling was applied to subjects with N covariates Z (indicated by zₙ where n=1 to N) and with a known structural model g. The true covariates were normalized to a mean of zero and a variance of 1 and then sampled from a distribution given by Equation 10a.

zₙ=ab{Normal(0,1)} (10)

A dataset of observations from 200 subjects was then generated using the NLME simulation methodologies in Pumas. The UENLME model was trained on the true values of only a percental P of the covariates. The dynamical model was assumed to be known matching the true dynamical model from the NLME simulation, while the structural g model was chosen to be fully described by neural networks where the weights were interpreted as fixed effects.

The predictive power of the learned UENLME model was then tested against the original truth NLME model where only the same percentage P of covariates were able to be used in the patient-specific predictions. This was done for multiple different truth models. A first truth model contained N=1,000 covariates in a linear structural model, P=100% known covariates, with 3,000 subjects and with a one-compartment model for the dynamics f. A second truth model contained N=20 covariates in a linear structural model, P=100% known covariates, with 300 subjects and with an exponential model for the dynamics A third truth model contained N=6 covariates in a nonlinear structural g model with pᵢ being a vector having two dynamical parameters (M=2) p1ᵢ and p2ᵢ given by Equations 10b and 10c.

p1ᵢ=0.7+0.04z₃z₂+0.5z₅ (10b)

p2ᵢ=40+40z₁/(0.4+z₁)−11z₃z₂+7z₄² (10c)

The covariate z₆ was intentionally left out of the model to simulate how the method acts on a non-informative covariate, i.e., that the network learns to ignore its value. In this third truth model, P=100% known covariates, with 300 subjects and with a one-compartment model for the dynamics. A fourth truth model contained N=20 covariates in a linear structural model, P=20% known covariates, with 300 subjects and with a one-compartment model for the dynamics.

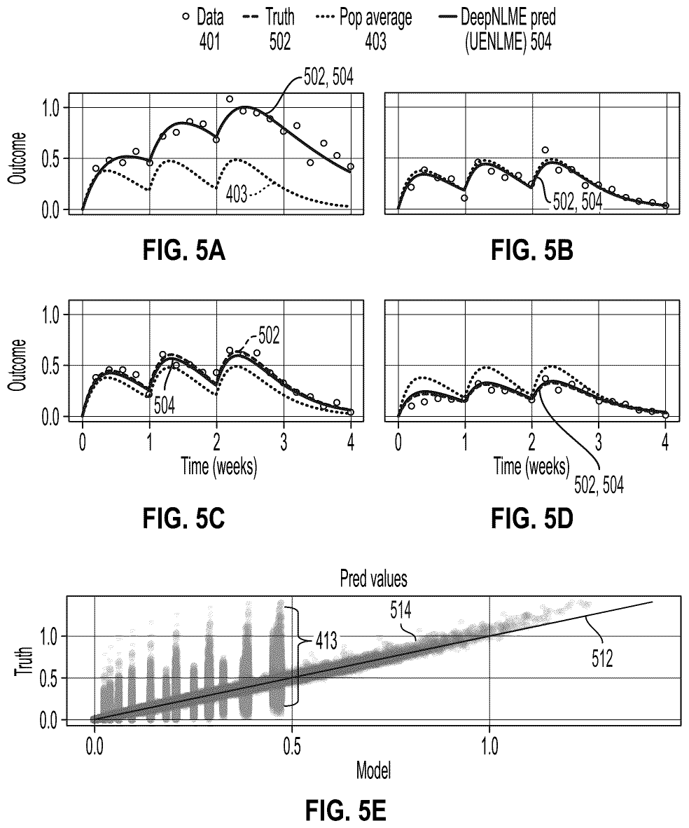

FIG. 5A through FIG. 5E are plots that illustrate example good fits to simulated data of drug concentration in a first truth model by a UENLME model trained on a training set when the trained UENLME model operates on a different test set, according to an embodiment. In each of FIG. 5A through FIG. 5D, respectively showing plots for each of four individuals, the horizontal axis is identical and indicates time from 0 to 4 weeks; and the vertical axis is identical and indicates outcome (normalized drug concentration) on a scale from 0 to 1.5. Data points 401 representing yᵢⱼ are indicated by dots, the first truth model values for uᵢⱼ are represented by traces 502, the UENLME model results after training are represented by traces 504. For comparison, the population average that does not consider the covariates among individuals is represented by trace 403 which is the same in each of FIG. 5A through FIG. 5D and thus not labeled in all. In FIG. 5E, the horizontal axis indicates estimates of normalized drug concentration (uᵢⱼ) from various sources and the vertical axis indicates the correct values of uᵢⱼ from the first truth model. The true values for uᵢⱼ are represented by trace 512. The population average that does not consider the covariates among individuals is represented by points 413. The UENLME model results after training are represented by points 514 that cluster nicely about the trace 512.

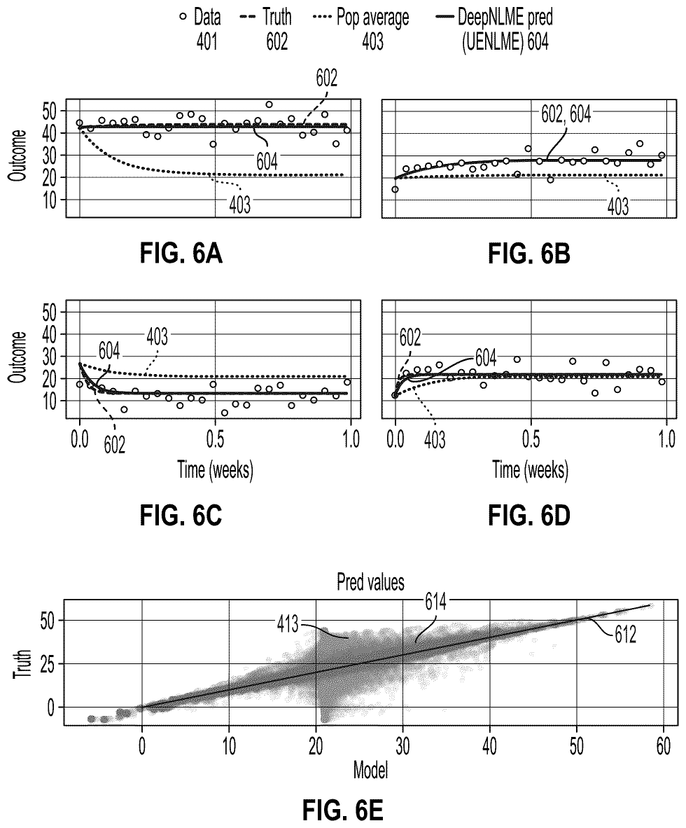

FIG. 6A through FIG. 6E are plots that illustrate example good fits similar to FIG. 5A through FIG. 5E but for a second truth model, according to an embodiment. In each of FIG. 6A through FIG. 6D, respectively showing plots for each of four individuals, the horizontal axis is identical and indicates time from 0 to 1 week; and the vertical axis is identical and indicates outcome (normalized drug concentration) on a scale from 0 to 50. Data points 401 representing yᵢⱼ are indicated by dots, the second truth model values for uᵢⱼ are represented by traces 602, the UENLME model results after training are represented by traces 604. For comparison, the population average that does not consider the covariates among individuals is represented by traces 403, which vary predominately based on initial concentrations. In FIG. 6E, the horizontal axis indicates estimates of normalized drug concentration (uᵢⱼ) from various sources and the vertical axis indicates the correct values of uᵢⱼ from the second truth model. The true values for uᵢⱼ are represented by trace 612. The population average that does not consider the covariates among individuals is represented by points 413. The UENLME model results after training are represented by points 614 that cluster nicely about the trace 612

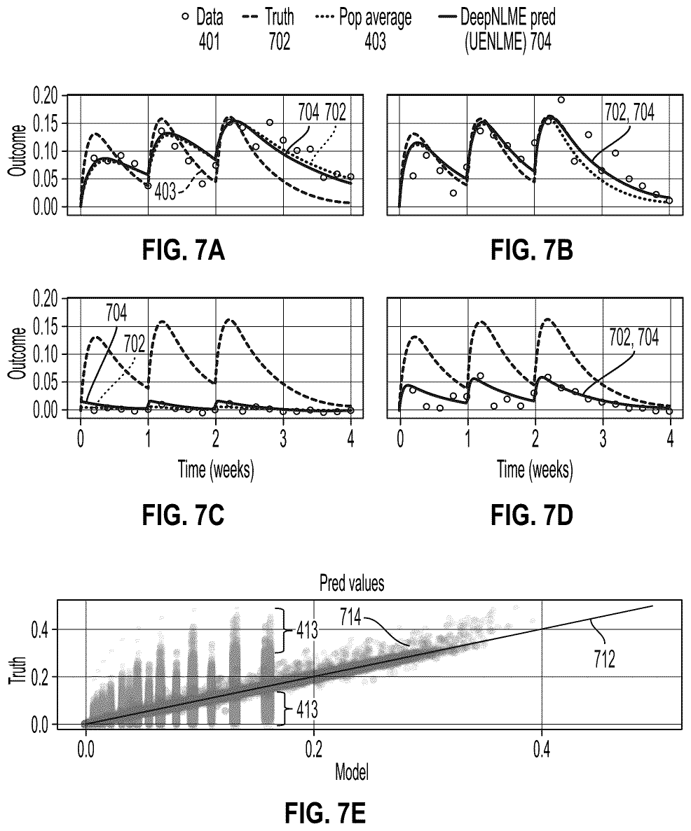

FIG. 7A through FIG. 7E are plots that illustrate example good fits similar to FIG. 5A through FIG. 5E but for a third truth model, according to an embodiment. In each of FIG. 7A through FIG. 7D, respectively showing plots for each of four individuals, the horizontal axis is identical and indicates time from 0 to 4 weeks; and the vertical axis is identical and indicates outcome (normalized drug concentration) on a scale from 0 to 0.20. Data points 401 representing yᵢⱼ are indicated by dots, the third truth model values for uᵢⱼ are represented by traces 702, the UENLME model results after training are represented by traces 704. For comparison, the population average that does not consider the covariates among individuals is represented by trace 403 which is the same in each of FIG. 7A through FIG. 7D and thus not labeled in all. In FIG. 7E, the horizontal axis indicates estimates of normalized drug concentration (uᵢⱼ) from various sources and the vertical axis indicates the correct values of uᵢⱼ from the third truth model. The true values for uᵢⱼ are represented by trace 712. The population average that does not consider the covariates among individuals is represented by points 413. The UENLME model results after training are represented by points 714 that cluster nicely about the trace 712.

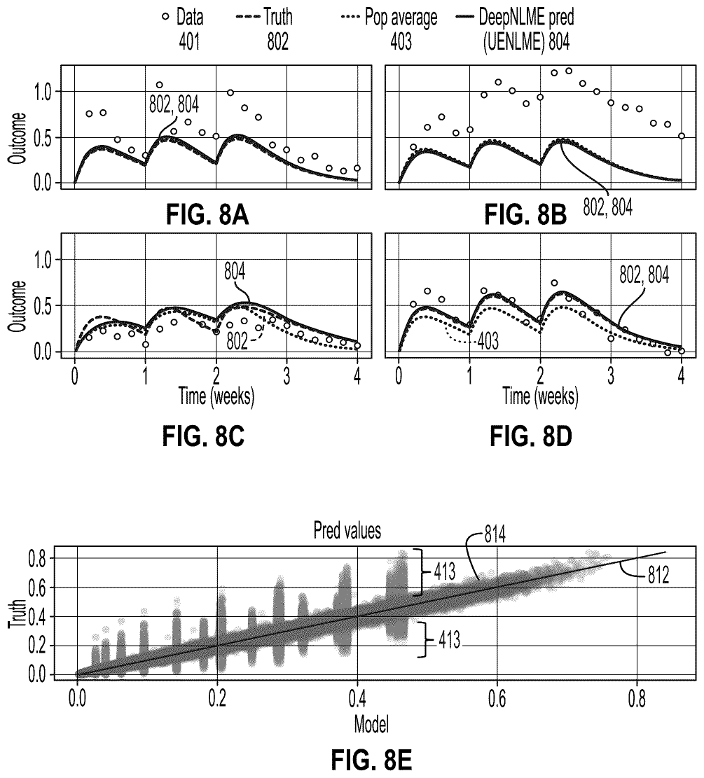

FIG. 8A through FIG. 8E are plots that illustrate example good fits similar to FIG. 5A through FIG. 5E but for a fourth truth model, according to an embodiment. In each of FIG. 8A through FIG. 8D, respectively showing plots for each of four individuals, the horizontal axis is identical and indicates time from 0 to 4 weeks; and the vertical axis is identical and indicates outcome (normalized drug concentration) on a scale from 0 to 1.0. Data points 401 representing yᵢⱼ are indicated by dots, the fourth truth model values for uᵢⱼ are represented by traces 802, the UENLME model results after training are represented by traces 804. For comparison, the population average that does not consider the covariates among individuals is represented by trace 403 which is the same in each of FIG. 8A through FIG. 8D and thus not labeled in all. The disagreements are relatively large in FIG. 8A and FIG. 8B because only 20% of the predictive covariates were given, so most of the patient-specific effects were not captured in the data given to the model. Those tests show that a UENLME does not have any noticeably more adverse effects to small data than standard NLME modeling, in fact the UENLME ends up giving a similar predictions. In FIG. 8E, the horizontal axis indicates estimates of normalized drug concentration (uᵢⱼ) from various sources and the vertical axis indicates the correct values of uᵢⱼ from the third truth model. The true values for uᵢⱼ are represented by trace 812. The population average that does not consider the covariates among individuals is represented by points 413. The UENLME model results after training are represented by points 814 that cluster nicely about the trace 812.

The results shown in FIG. 5A through FIG. 8E demonstrate that the UENLME model learns a structural model g that very closely matches the predictions of the original NLME model on a subject not included in the training data for each of the 4 truth models. This shows the UENLME model has successfully trained to give accurate patient-specific drug concentration and response information from partial and noisy information.

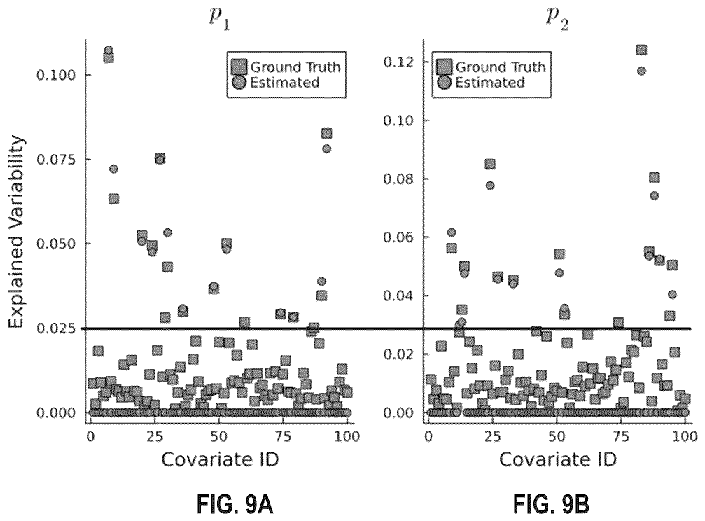

The use and effectiveness of the optional symbolic regression step, was demonstrated by performing a LASSO STLQS symbolic regression by sampling input output pairs on the trained neural network approximation to g. This symbolic regression approach involved a chosen dictionary (though not all symbolic regression methods do), which was chosen to be a linear function, and thus the output of this step was a linear model approximation for g which maximizes the population likelihood. N=100 covariates were considered, including most based on features manually derived from medical imagery. FIG. 9A and FIG. 9B are plots that compare the actual contributions of 100 covariates in vector Z for a structural model to the estimated contributions of those covariates using a symbolic regression derived from the UENLME model, for two dynamic parameters p1 and p2, respectively [, according to an embodiment. The horizontal axis in each indicates the covariate index n, varying from 1 to N=100. The vertical axis in each indicates the amount of that dynamic parameter variability explained by each covariate. The solid squares indicate the values from the truth model (called “Ground Truth” in the figures) and the solid circles indicate the values of coefficients of the linear model derived from the UENLME model that was previously fit to the training set (called “Estimated” in the figures).

FIG. 9A and FIG. 9B show that almost all of the covariates whose variability significantly affects g (e.g., with explained variability values greater than 0.025) were successfully captured with appropriate coefficients in the linear model, while the other coefficients were given zero coefficients in the linear model. The resulting simplified linear model is given by Equations 10d and 10e, where θ1 and θ2 represent the population average, covariate independent, values of the two dynamical parameters.

p1ᵢ=θ1−0.558+0.72z₉+0.047z₂₄+0.03z₇₄+0.051z₂₀+0.053z₃₀+0.075z₂₇+0.107z₇+0.028z₇₉+0.031z₃₆+0.037z₄₈+0.048z₅₃+0.039z₉₀+0.078z₉₂ (10d)

p2ᵢ=θ2−0.598+0.62z₉+0.03z₁₂+0.031z₁₃+0.036z₅₃+0.048z₁₄+0.04z₉₅+0.046z₂₇+0.078z₂₄+0.044z₃₃+0.048z₅₁+0.117z₈₃+0.052z₉₀+0.054z₈₆+0.074z₈₈ (10e)

This demonstrates that the techniques described here can be used to identify the most explanatory covariates from a large dataset, for example automatically deriving the most predictive elements from -omics data, SNP arrays, and EHR for predicting patient-specific dosing outcomes and automating the clinical trial design process.

In one embodiment, UENLME modeling was applied for a case where the structural model g and the dynamical model f were both assumed to be unknown. The structural model g was assumed to be generating reaction rates of a dynamical model which must be positive, and thus a semi-structural form was chosen to guarantee positivity of the resulting predictions. This form is given by Equation 11a.

log(pᵢ)=log(θ)+U₁(mean(Zᵢ),ϕ₁) (11a)

where mean(Zᵢ) is a vector of the population means for each covariate, z₁ through z_{N}. Here U1 was chosen to be a neural network. Similarly, the dynamical model f was chosen to be fully described by a neural network, e.g., a simplified version of Equation 6a, above, given now by Equation 11b.

u′=U₂(u,pᵢ,ϕ₂) (11b)

The weights of both of these neural networks, ϕ₁ and ϕ₂, were interpreted as fixed effects. The training process took 15 minutes using automatic differentiation for the gradient calculations.

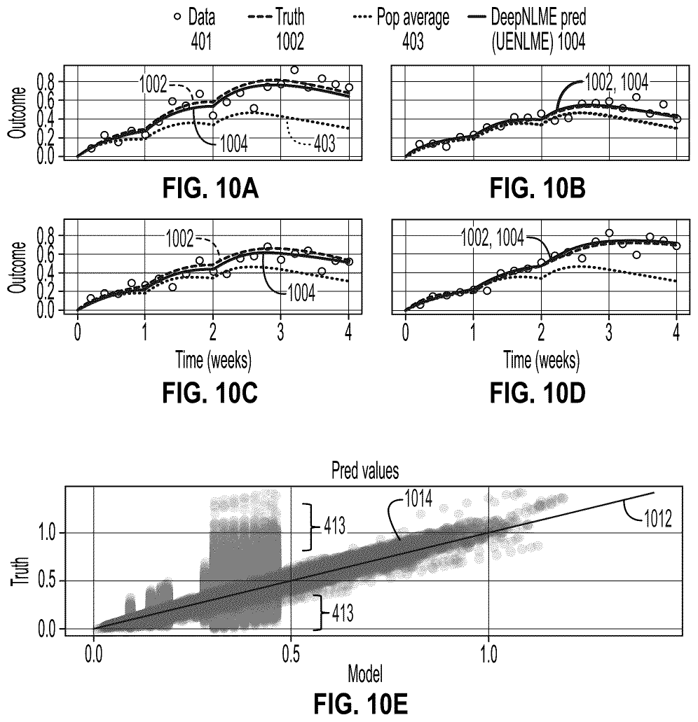

FIG. 10A through FIG. 10E are plots that illustrate example good fits similar to FIG. 5A through FIG. 5E but for universal approximator spanning both a structural function g and a dynamical function f, according to an embodiment. In each of FIG. 10A through FIG. 10D, respectively showing plots for each of four individuals, the horizontal axis is identical and indicates time from 0 to 4 weeks; and the vertical axis is identical and indicates outcome (normalized drug concentration) on a scale from 0 to 0.8. Data points 401 representing yᵢⱼ are indicated by dots, the true values for uᵢⱼ are represented by traces 1002, the results from the UENLME model of Equations 11a and 11b, after training, are represented by traces 1004. For comparison, the population average that does not consider the covariates among individuals is represented by trace 403 which is the same in each of FIG. 10A through FIG. 10D and thus not labeled in all. In FIG. 10E, the horizontal axis indicates estimates of normalized drug concentration (uᵢⱼ) from various sources and the vertical axis indicates the correct values of uᵢⱼ from the truth model. The true values for uᵢⱼ are represented by trace 1012. The population average that does not consider the covariates among individuals is represented by points 413. The UENLME model results after training are represented by points 1014 that cluster nicely about the trace 1012.

The results in FIG. 10A through FIG. 10E show that this model form of Equations 11a and 11b combining universal approximators in each of two models of the NLME was also able to sufficiently learn an NLME model that could predict patient-specific drug concentrations and responses on a subject (set of covariates) that was not in the training data.

To demonstrate the ability for UENLME to be used for optimizing dosage regimens, the ability for the trained UENLME model to predict patient drug concentrations over time and patient response under dosage regimens which were different from the one used in the training data was tested. In this case the UENLME model included both f and g as neural networks, and all of the weights were considered fixed effects. The training data was time series per-patient. The training dataset was for patients given 3 doses over 4 weeks. The predictions show the UENLME model and the original NLME data generating model, each with 5 and 2 doses over four weeks. The patients in the prediction set were different from those in the training set, essentially with new values Zi of the covariates.

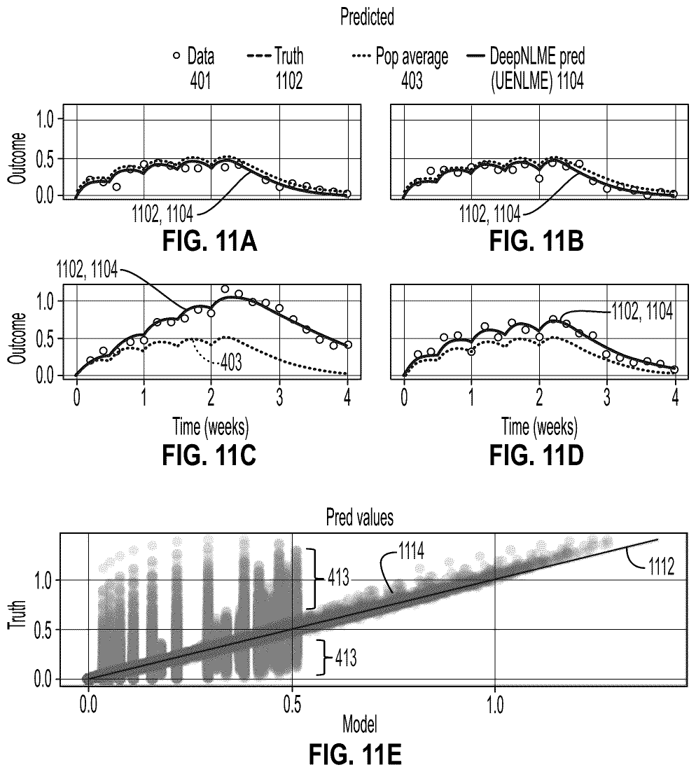

FIG. 11A through FIG. 11E are plots that illustrate example good fits similar to FIG. 5A through FIG. 5E but for a different dosing regimen than in a training set, according to an embodiment. In each of FIG. 11A through FIG. 11D, respectively showing plots for each of four individuals, the horizontal axis is identical and indicates time from 0 to 4 weeks; and the vertical axis is identical and indicates outcome (normalized drug concentration) on a scale from 0 to 1.0. Data points 401 representing yᵢⱼ are indicated by dots, the truth model values for uᵢⱼ are represented by traces 1102, the UENLME model results after training are represented by traces 1104. For comparison, the population average that does not consider the covariates among individuals is represented by trace 403 which is the same in each of FIG. 11A through FIG. 11D and thus not labeled in all. In FIG. 11E, the horizontal axis indicates estimates of normalized drug concentration (uᵢⱼ) from various sources and the vertical axis indicates the correct values of uᵢⱼ from the truth model. The true values for uᵢⱼ are represented by trace 1112. The population average that does not consider the covariates among individuals is represented by points 413. The UENLME model results after training are represented by points 1114 that cluster nicely about the trace 1112.

FIG. 11A through FIG. 11E illustrates how the UENLME model trained using data applying three doses over 4 weeks is able to accurately predict how the patient-specific responses change as the dosage regimens change to five doses in four weeks.

### 3. Computational Hardware Overview

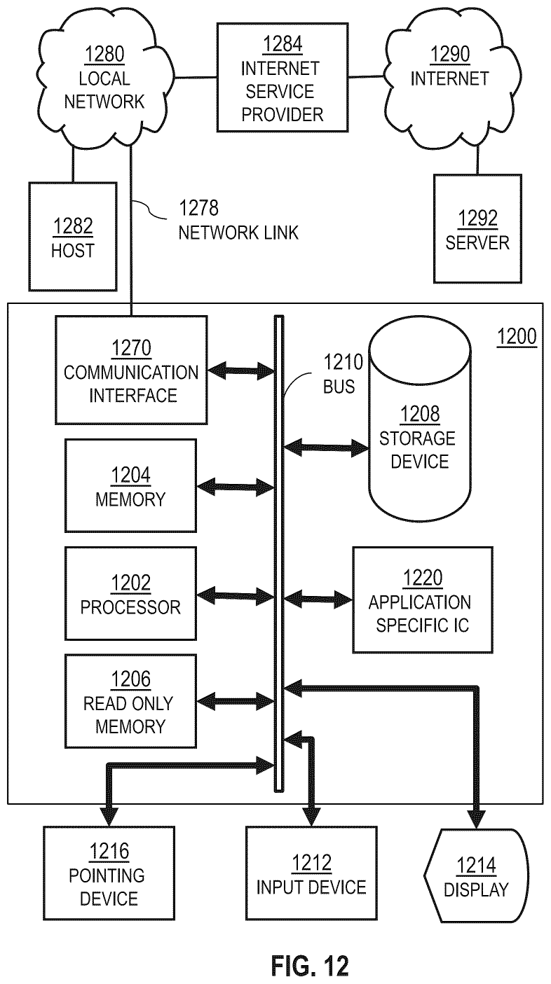

FIG. 12 is a block diagram that illustrates a computer system 1200 upon which an embodiment of the invention may be implemented. Computer system 1200 includes a communication mechanism such as a bus 1210 for passing information between other internal and external components of the computer system 1200. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 1200, or a portion thereof, constitutes a means for performing one or more steps of one or more methods described herein.

A sequence of binary digits constitutes digital data that is used to represent a number or code for a character. A bus 1210 includes many parallel conductors of information so that information is transferred quickly among devices coupled to the bus 1210. One or more processors 1202 for processing information are coupled with the bus 1210. A processor 1202 performs a set of operations on information. The set of operations include bringing information in from the bus 1210 and placing information on the bus 1210. The set of operations also typically include comparing two or more units of information, shifting positions of units of information, and combining two or more units of information, such as by addition or multiplication. A sequence of operations to be executed by the processor 1202 constitutes computer instructions.

Computer system 1200 also includes a memory 1204 coupled to bus 1210. The memory 1204, such as a random access memory (RAM) or other dynamic storage device, stores information including computer instructions. Dynamic memory allows information stored therein to be changed by the computer system 1200. RAM allows a unit of information stored at a location called a memory address to be stored and retrieved independently of information at neighboring addresses. The memory 1204 is also used by the processor 1202 to store temporary values during execution of computer instructions. The computer system 1200 also includes a read only memory (ROM) 1206 or other static storage device coupled to the bus 1210 for storing static information, including instructions, that is not changed by the computer system 1200. Also coupled to bus 1210 is a non-volatile (persistent) storage device 1208, such as a magnetic disk or optical disk, for storing information, including instructions, that persists even when the computer system 1200 is turned off or otherwise loses power.

Information, including instructions, is provided to the bus 1210 for use by the processor from an external input device 1212, such as a keyboard containing alphanumeric keys operated by a human user, or a sensor. A sensor detects conditions in its vicinity and transforms those detections into signals compatible with the signals used to represent information in computer system 1200. Other external devices coupled to bus 1210, used primarily for interacting with humans, include a display device 1214, such as a cathode ray tube (CRT) or a liquid crystal display (LCD), for presenting images, and a pointing device 1216, such as a mouse or a trackball or cursor direction keys, for controlling a position of a small cursor image presented on the display 1214 and issuing commands associated with graphical elements presented on the display 1214.

In the illustrated embodiment, special purpose hardware, such as an application specific integrated circuit (IC) 1220, is coupled to bus 1210. The special purpose hardware is configured to perform operations not performed by processor 1202 quickly enough for special purposes. Examples of application specific ICs include graphics accelerator cards for generating images for display 1214, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1200 also includes one or more instances of a communications interface 1270 coupled to bus 1210. Communication interface 1270 provides a two-way communication coupling to a variety of external devices that operate with their own processors, such as printers, scanners and external disks. In general the coupling is with a network link 1278 that is connected to a local network 1280 to which a variety of external devices with their own processors are connected. For example, communication interface 1270 may be a parallel port or a serial port or a universal serial bus (USB) port on a personal computer. In some embodiments, communications interface 1270 is an integrated services digital network (ISDN) card or a digital subscriber line (DSL) card or a telephone modem that provides an information communication connection to a corresponding type of telephone line. In some embodiments, a communication interface 1270 is a cable modem that converts signals on bus 1210 into signals for a communication connection over a coaxial cable or into optical signals for a communication connection over a fiber optic cable. As another example, communications interface 1270 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 1270 sends and receives electrical, acoustic or electromagnetic signals, including infrared and optical signals, that carry information streams, such as digital data.

The term computer-readable medium is used herein to refer to any medium that participates in providing information to processor 1202, including instructions for execution. Such a medium may take many forms, including, but not limited to, non-volatile media, volatile media and transmission media. Non-volatile media include, for example, optical or magnetic disks, such as storage device 1208. Volatile media include, for example, dynamic memory 1204. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 1202, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 1202, except for carrier waves and other signals.

Logic encoded in one or more tangible media includes one or both of processor instructions on a computer-readable storage media and special purpose hardware, such as ASIC 1220.

Network link 1278 typically provides information communication through one or more networks to other devices that use or process the information. For example, network link 1278 may provide a connection through local network 1280 to a host computer 1282 or to equipment 1284 operated by an Internet Service Provider (ISP). ISP equipment 1284 in turn provides data communication services through the public, world-wide packet-switching communication network of networks now commonly referred to as the Internet 1290. A computer called a server 1292 connected to the Internet provides a service in response to information received over the Internet. For example, server 1292 provides information representing video data for presentation at display 1214.

The invention is related to the use of computer system 1200 for implementing the techniques described herein. According to one embodiment of the invention, those techniques are performed by computer system 1200 in response to processor 1202 executing one or more sequences of one or more instructions contained in memory 1204. Such instructions, also called software and program code, may be read into memory 1204 from another computer-readable medium such as storage device 1208. Execution of the sequences of instructions contained in memory 1204 causes processor 1202 to perform the method steps described herein. In alternative embodiments, hardware, such as application specific integrated circuit 1220, may be used in place of or in combination with software to implement the invention. Thus, embodiments of the invention are not limited to any specific combination of hardware and software.

The signals transmitted over network link 1278 and other networks through communications interface 1270, carry information to and from computer system 1200. Computer system 1200 can send and receive information, including program code, through the networks 1280, 1290 among others, through network link 1278 and communications interface 1270. In an example using the Internet 1290, a server 1292 transmits program code for a particular application, requested by a message sent from computer 1200, through Internet 1290, ISP equipment 1284, local network 1280 and communications interface 1270. The received code may be executed by processor 1202 as it is received, or may be stored in storage device 1208 or other non-volatile storage for later execution, or both. In this manner, computer system 1200 may obtain application program code in the form of a signal on a carrier wave.

Various forms of computer readable media may be involved in carrying one or more sequence of instructions or data or both to processor 1202 for execution. For example, instructions and data may initially be carried on a magnetic disk of a remote computer such as host 1282. The remote computer loads the instructions and data into its dynamic memory and sends the instructions and data over a telephone line using a modem. A modem local to the computer system 1200 receives the instructions and data on a telephone line and uses an infra-red transmitter to convert the instructions and data to a signal on an infra-red a carrier wave serving as the network link 1278. An infrared detector serving as communications interface 1270 receives the instructions and data carried in the infrared signal and places information representing the instructions and data onto bus 1210. Bus 1210 carries the information to memory 1204 from which processor 1202 retrieves and executes the instructions using some of the data sent with the instructions. The instructions and data received in memory 1204 may optionally be stored on storage device 1208, either before or after execution by the processor 1202.

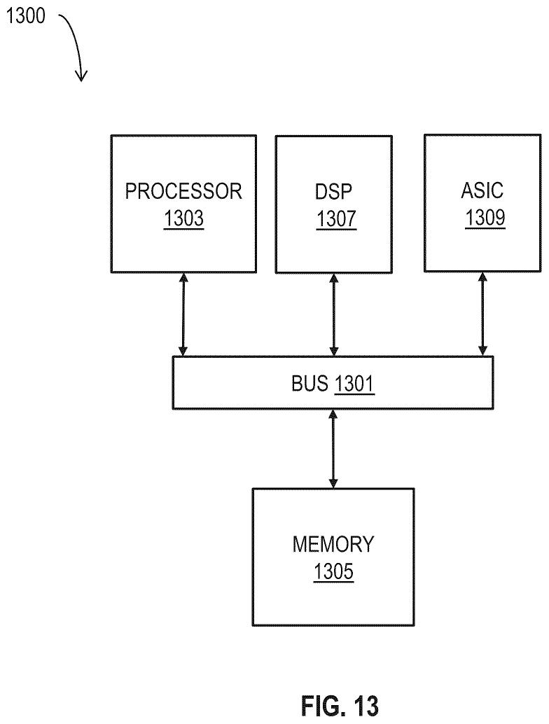

FIG. 13 illustrates a chip set 1300 upon which an embodiment of the invention may be implemented. Chip set 1300 is programmed to perform one or more steps of a method described herein and includes, for instance, the processor and memory components described with respect to FIG. 12 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1300, or a portion thereof, constitutes a means for performing one or more steps of a method described herein.

In one embodiment, the chip set 1300 includes a communication mechanism such as a bus 1301 for passing information among the components of the chip set 1300. A processor 1303 has connectivity to the bus 1301 to execute instructions and process information stored in, for example, a memory 1305. The processor 1303 may include one or more processing cores with each core configured to perform independently. A multi-core processor enables multiprocessing within a single physical package. Examples of a multi-core processor include two, four, eight, or greater numbers of processing cores. Alternatively, or in addition, the processor 1303 may include one or more microprocessors configured in tandem via the bus 1301 to enable independent execution of instructions, pipelining, and multithreading. The processor 1303 may also be accompanied with one or more specialized components to perform certain processing functions and tasks such as one or more digital signal processors (DSP) 1307, or one or more application-specific integrated circuits (ASIC) 1309. A DSP 1307 typically is configured to process real-world signals (e.g., sound) in real time independently of the processor 1303. Similarly, an ASIC 1309 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1303 and accompanying components have connectivity to the memory 1305 via the bus 1301. The memory 1305 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1305 also stores the data associated with or generated by the execution of one or more steps of the methods described herein.

### 4. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 5. References

The following documents are hereby incorporated by reference as if fully set forth herein except for terminology inconsistent with that used above.
Andrzej Lasota and Michael C. Mackey. Chaos, Fractals, and Noise: Stochastic Aspects of Dynamics Springer Science & Business Media, Nov. 27, 2013. 481 pp. isbn: 978-1-4612-4286-4.
Andrew Leonard. “Probabilistic Methods for Decision Making in Precision Airdrop”. GA Tech, Jan. 15, 2019. 141 pp.
Christopher Rackauckas and Qing Nie. “DifferentialEquations.Jl—A Performant and Feature-Rich Ecosystem for Solving Differential Equations in Julia”. In: J. Open Res. Softw. 5.1 (1 May 25, 2017), p. 15. issn: 2049-9647. doi: 10.5334/jors.151.
Andrea Yeong Weiße. “Global Sensitivity Analysis of Ordinary Differential Equations: Adaptive Density Propagation Using Approximate Approximations”. In: (2009). Accepted: 2018-06-07T22:43:56Z. at subdomain dx of domain doi of super-domain org in folder 10.17169 and file refubium-13784.
Gerlach, A. R., Leonard, A., Rogers, J., & Rackauckas, C. (2020) “The Koopman Expectation: An Operator Theoretic Method for Efficient Analysis and Optimization of Uncertain Hybrid Dynamical Systems,” available at domain arxiv of super-domain org in folder abs at link 2008.08737v1 to file 2008.08737v1.pdf.
Christopher Rackauckas, Yingbo Ma, Julius Martensen, Collin Warner, Kirill Zubov, Rohit Supekar, Dominic Skinner, Ali Ramadhan, Alan Edelman, “Universal Differential Equations for Scientific Machine Learning, at domain,” arXiv in org in folder abs in file 2001.04385, January 2020.
Raissia, M., P. Perdikaris, and G. E. Karniadakisa “Physics-informed neural networks: A deep learning framework for solving forward and inverse problems involving nonlinear partial differential equations,” Journal of Computational Physics, Volume 378, 1 Feb. 2019, Pages 686-707.
Miles Cranmer, “Distributed High-Performance symbolic regression in Julia,” at domain github of com in folder MilesCranmer in file SymbolicRegression.jl, November 2021.
Ricky T. Q. Chen, Yulia Rubanova, Jesse Bettencourt, David Duvenaud, “Neural Ordinary Differential Equations” at domain arXiv of org in folder abs file 1806.07366, December 2019.
Suyong Kim, Weiqi Ji, Sili Deng, Yingbo Ma, and Christopher Rackauckas, “Stiff neural ordinary differential equations,” Chaos 31, 093122 (2021), 20 Sep. 2021.
Steven L. Brunton, Joshua L. Proctor, and J. Nathan Kutz, “Discovering governing equations from data by sparse identification of nonlinear dynamical systems” Proc. Natl. Acad. Sci. (PNAS) Apr. 12, 2016 113 (15) 3932-3937.
Rackauckas, C; Edelman, A; Fischer, K; Innes, M; Saba, E; Shah, V B; Tebbutt, “Generalized physics-informed learning through language-wide differentiable programming,” CEUR Workshop Proceedings, 2587. 2020 available on the World Wide Web in subdomain dspace of domain mit in org in folder handle subfolder 1721.1 file 137320.

What is claimed is:

1. A method executed automatically on a processor for generating a dosing protocol for an individual subject, the method comprising:
receiving first data that indicates, for a dose response to a medicament, a non-linear mixed effects (NLME) model of a population with at least one distribution parameter characterizing variations in the population based on an observable property of individuals within the population wherein at least one of a structural model or a dynamical model of the NLME model is based on training weights of a universal approximator on a least a subset of the population;
evaluating for a candidate dose regimen an expected response by a subject based on the NLME model and one or more properties of the subject; and
when the expected response is therapeutic, causing the candidate dose regime of the medicament to be administered to the subject.

2. The method of claim 1 wherein the universal approximator is a neural network.

3. The method of claim 1 wherein said training weights of the universal approximator is confined to training fixed effect weights of the universal approximator.

4. The method of claim 1 wherein said training weights of the universal approximator includes training random effect weights of the universal approximator to represent deviations of an individual from other individuals with the same observable property.

5. The method of claim 3 wherein the structural model or the dynamical model of the NLME model based on said training fixed-effect weights is implemented as a linear or non-linear model derived from the fixed-effect weights of the universal approximator.

6. A non-transitory computer-readable medium carrying one or more sequences of instructions for generating a dosing protocol for an individual, wherein execution of the one or more sequences of instructions by one or more processors causes the one or more processors to:
receive first data that indicates, for a dose response to a medicament, a non-linear mixed effects (NLME) model of a population with at least one distribution parameter characterizing variations in the population based on an observable property of individuals within the population wherein at least one of a structural model or a dynamical model of the NLME model is based on training weights of a universal approximator on a least a subset of the population;
evaluate for a candidate dose regimen an expected response by a subject based on the NLME model and one or more properties of the subject; and
when the expected response is therapeutic, cause the candidate dose regime of the medicament to be administered to the subject.

7. The computer-readable medium of claim 6 wherein the universal approximator is a neural network.

8. The computer-readable medium of claim 6 wherein said training weights of the universal approximator is confined to training fixed effect weights of the universal approximator.

9. The computer-readable medium of claim 6 wherein said training weights of the universal approximator includes training random effect weights of the universal approximator to represent deviations of an individual from other individuals with the same observable property.

10. The computer-readable medium of claim 8 wherein the structural model or the dynamical model of the NLME model based on said training fixed-effect weights is implemented as a linear or non-linear model derived from the fixed-effect weights of the universal approximator.

11. An apparatus for generating a dosing protocol for an individual, the apparatus comprising:
at least one processor; and
at least one memory including one or more sequences of instructions,
the at least one memory and the one or more sequences of instructions configured to, with the at least one processor, cause the apparatus to:
receive first data that indicates, for a dose response to a medicament, a non-linear mixed effects (NLME) model of a population with at least one distribution parameter characterizing variations in the population based on an observable property of individuals within the population wherein at least one of a structural model or a dynamical model of the NLME model is based on training weights of a universal approximator on a least a subset of the population;
evaluate for a candidate dose regimen an expected response by a subject based on the NLME model and one or more properties of the subject; and
when the expected response is therapeutic, cause the candidate dose regime of the medicament to be administered to the subject.

12. The apparatus of claim 11 wherein the universal approximator is a neural network.

13. The apparatus of claim 11 wherein said training weights of the universal approximator is confined to training fixed effect weights of the universal approximator.

14. The apparatus of claim 11 wherein said training weights of the universal approximator includes training random effect weights of the universal approximator to represent deviations of an individual from other individuals with the same observable property.

15. The apparatus of claim 13 wherein the structural model or the dynamical model of the NLME model based on said training fixed-effect weights is implemented as a linear or non-linear model derived from the fixed-effect weights of the universal approximator.
